(19) United States
(12) Patent Application Publication
(10) Pub. No.: US 2009/0118294 A1
(43) Pub. Date: May 7, 2009

(54) COMPOSITIONS AND METHODS RELATED TO HEART FAILURE

(76) Inventors: Manuel Worcel, Boston, MA (US); Michael L. Sabolinski, Franklin, MA (US)

Correspondence Address: WILMERHALE/NITROMED, 1875 PENNSYLVANIA AVE, NW, WASHINGTON, DC, 20006, US

(21) Appl. No.: 12/270,625

(22) Filed: Nov. 13, 2008

Related U.S. Application Data
(63) Continuation of application No. 11/182,886, filed on Jul. 18, 2005.
(60) Provisional application No. 60/588,390, filed on Jul. 16, 2004.
(60) Provisional application No. 60/600,354, filed on Aug. 11, 2004.
(60) Provisional application No. 60/610,901, filed on Sep. 20, 2004.
(60) Provisional application No. 60/622,781, filed on Oct. 29, 2004.
(60) Provisional application No. 60/625,056, filed on Nov. 5, 2004.
(60) Provisional application No. 60/669,925, filed on Apr. 11, 2005.
(60) Provisional application No. 60/684,892, filed on May 26, 2005.
(60) Provisional application No. 60/689,520, filed on Jun. 13, 2005.

(51) Int. Cl. A61K 31/50

(52) U.S. Cl. 514/248

(57) ABSTRACT

The invention provides methods for reducing the total number of days a patient with heart failure spends in the hospital for heart failure for a single hospital stay (i.e., reducing the duration of a single hospital stay for a patient with heart failure); reducing the total number of days a patient spends in the hospital for heart failure for multiple hospital stays (i.e., two or more hospital stays); reducing the number of hospital admissions for heart failure; and reducing mortality and reducing hospitalizations for heart failure (e.g., the total number of days in the hospital and/or the number of hospital visits); by administering to a patient a therapeutically effective amount of (i) a hydralazine compound or pharmaceutically acceptable salt thereof, (ii) isosorbide dinitrate and/or isosorbide mononitrate, and (iii) optionally at least one angiotensin converting enzyme inhibitors, β-adrenergic antagonists, angiotensin II antagonists, aldosterone antagonists, cardiac glucosides (digitalis), or diuretic compounds.

### RELATED APPLICATIONS

This application is a continuation application of U.S. application Ser. No. 11/182,886 filed Jul. 18, 2005, which claims priority under 35 USC § 119 to U.S. Application No. 60/588,390 filed Jul. 16, 2004, U.S. Application No. 60/600,354 filed Aug. 11, 2004, U.S. Application No. 60/610,901 filed Sep. 20, 2004, U.S. Application No. 60/622,781 filed Oct. 29, 2004, U.S. Application No. 60/625,056 filed Nov. 5, 2004, U.S. Application No. 60/669,925 filed Apr. 11, 2005, U.S. Application No. 60/684,892 filed May 26, 2005, and U.S. Application No. 60/689,520 filed Jun. 13, 2005; the disclosures of each of which are incorporated by reference herein in their entirety.

### FIELD OF THE INVENTION

The invention provides methods for (a) reducing hospitalizations related to heart failure; (b) increasing the left ventricular ejection fraction in a heart failure patient; (c) treating a sexual dysfunction (d) treating a headache in a heart failure patient by administering a non-steroidal antiinflammatory compound; (e) treating a heart failure patient who has a history of hypertension (but who is not currently diagnosed with hypertension); (f) improving the quality of life in a heart failure patient based on the Minnesota Living with Heart Failure Quality of Life questionnaire; and (g) decreasing the levels of B-type natriuretic peptide in a patient in need thereof comprising administering a therapeutically effective amount of (i) at least one hydralazine compound or a pharmaceutically acceptable salt thereof, (ii) isosorbide dinitrate and/or isosorbide mononitrate, and (iii) optionally the best current therapy for the cardiovascular disease being treated.

### BACKGROUND OF THE INVENTION

The decline in cardiovascular morbidity and mortality in the United States over the past three decades has been the result of significant advances in research on cardiovascular disease mechanisms and therapeutic strategies. The incidence and prevalence of myocardial infarction and death from myocardial infarction, as well as that from cerebrovascular accident, have decreased significantly over this period largely owing to advances in prevention, early diagnosis, and treatment of these very common diseases.

Congestive heart failure (CHF) is a clinical syndrome involving cardiac and peripheral abnormalities that produce morbidity and shortened life span. This syndrome is now the leading cause of hospitalization in individuals older than age 65 and is a major contributor to the escalation of heath care costs.

There is a need in the art for new and more effective compositions and methods for reducing mortality associated with heart failure, in improving oxygen consumption, quality of life and/or exercise tolerance in patients and for prolonging time to hospitalization. The invention is directed to these, as well as other, important ends.

### SUMMARY OF THE INVENTION

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction; (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient a therapeutically effective amount of (i) a hydralazine compound or pharmaceutically acceptable salt thereof, (ii) isosorbide dinitrate and/or isosorbide mononitrate, and (iii) optionally at least one compound selected from the group consisting of angiotensin converting enzyme inhibitors, β-adrenergic antagonists, angiotensin II antagonists, aldosterone antagonists, cardiac glucosides (digitalis), and diuretic compounds. In one embodiment, the patients are categorized as New York Heart Association heart failure functional classification I, II, III or IV; preferably III or IV. In another embodiment the patient is a black patient.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient a therapeutically effective amount of hydralazine hydrochloride and isosorbide dinitrate, and, optionally, at least one compound selected from the group consisting of angiotensin converting enzyme inhibitors, β-adrenergic antagonists, angiotensin II antagonists, aldosterone antagonists, cardiac glycosides, and diuretic compounds. In one embodiment, the patient is categorized as New York Heart Association heart failure functional classification I, II, III or IV; preferably III or IV. In another embodiment the patient is a black patient.

The invention provides methods for prolonging time to hospitalization for heart failure in a patient in need thereof comprising administering to the patient a therapeutically effective amount of (i) a hydralazine compound or pharmaceutically acceptable salt thereof, (ii) isosorbide dinitrate and/or isosorbide mononitrate, and (iii) optionally at least one compound selected from the group consisting of angiotensin converting enzyme inhibitors, β-adrenergic antagonists, angiotensin II antagonists, aldosterone antagonists, cardiac glycosides, and diuretic compounds. In one embodiment, the patients are categorized as New York Heart Association heart failure functional classification I, II, III or IV; preferably III or IV. In another embodiment the patient is a black patient.

These and other aspects of the invention are described in detail herein.

### BRIEF DESCRIPTION OF THE FIGURES

FIG. 1 summarizes the effect of hydralazine hydrochloride and isosorbide dinitrate on the composite score in subgroups.

FIG. 2 shows the Kaplan-Meier time-to-event curves for all-cause mortality

FIG. 3 shows the hazard ratios and 95% confidence intervals for hydralazine hydrochloride and isosorbide dinitrate on all-cause mortality in subgroups.

FIG. 4 shows the Kaplan-Meier time-to-event curves for first hospitalization for heart failure.

FIG. 5 shows the Kaplan-Meier time-to-first event analysis of all-cause mortality or hospitalization for heart failure.

FIG. 6 shows the mean change in the Minnesota living with heart failure questionnaire overall score at each visit and at the endpoint.

### DETAILED DESCRIPTION OF THE INVENTION

As used throughout the disclosure, the following terms, unless otherwise indicated, shall be understood to have the following meanings.

“Patient” refers to animals, preferably mammals, most preferably humans, and includes males and females.

“Black” refers to a person of African descent or an African-American person. A person may be African-American or black if he/she designates himself/herself as such.

“Therapeutically effective amount” refers to the amount of the compound and/or composition that is effective to achieve its intended purpose.

“Endothelial dysfunction” refers to the impaired ability in any physiological processes carried out by the endothelium, in particular, production of nitric oxide regardless of cause. It may be evaluated by, such as, for example, invasive techniques, such as, for example, coronary artery reactivity to acetylcholine or methacholine, and the like, or by noninvasive techniques, such as, for example, blood flow measurements, brachial artery flow dilation using cuff occlusion of the arm above or below the elbow, brachial artery ultrasonography, imaging techniques, measurement of circulating biomarkers, such as, asymmetric dimethylarginine (ADMA), and the like. For the latter measurement the endothelial-dependent flow-mediated dialation will be lower in patients diagnosed with an endothelial dysfunction.

“Oxygen consumption” can be measured during a progressive maximal bicycle-ergometer exercise test taken while the expired air is collected continuously to monitor oxygen consumption. Dyspnea or fatigue typically occurs at a peak oxygen consumption of <25 ml per kilogram of body weight per minute. Patients with pulmonary diseases, obstructive valvular diseases and the like, tend to have a low oxygen consumption. An increase in a patient's oxygen consumption typically results in the patient's increased exercise tolerance and would imply that the patient would have an improved quality of life.

“Quality of life” refers to one or more of a person's ability to walk, climb stairs, do errands, work around the house, participate in recreational activities, and/or not requiring rest during the day, and/or the absence of sleeping problems or shortness of breath. The quality of life can be measured using the Minnesota Living with Heart Failure questionnaire. The questionnaire is self-administered after brief standardization instructions. The score is obtained by summing the ranks of the responses to each question.

“Sexual dysfunction” refers to and includes male erectile dysfunction and female sexual dysfunction. Sexual dysfunction includes, but is not limited to, for example, sexual pain disorders, sexual desire disorders, sexual arousal dysfunction, orgasmic dysfunction, dyspareunia, vaginismus, and the like.

“Angiotensin converting enzyme (ACE-I) inhibitor” refers to compounds that inhibit an enzyme which catalyzes the conversion of angiotensin I to angiotensin II. ACE inhibitors include, but are not limited to, amino acids and derivatives thereof, peptides, including di- and tri-peptides, and antibodies to ACE which intervene in the renin-angiotensin system by inhibiting the activity of ACE thereby reducing or eliminating the formation of the pressor substance angiotensin II.

“Angiotensin II antagonists” refers to compounds which interfere with the function, synthesis or catabolism of angiotensin II. Angiotensin II antagonists include peptide compounds and non-peptide compounds, including, but not limited to, angiotensin II antagonists, angiotensin II receptor antagonists, agents that activate the catabolism of angiotensin II, and agents that prevent the synthesis of angiotensin I from angiotensin II. The renin-angiotensin system is involved in the regulation of hemodynamics and water and electrolyte balance. Factors that lower blood volume, renal perfusion pressure, or the concentration of sodium in plasma tend to activate the system, while factors that increase these parameters tend to suppress its function.

“Carriers” or “vehicles” refers to carrier materials suitable for compound administration and include any such material known in the art such as, for example, any liquid, gel, solvent, liquid diluent, solubilizer, or the like, which is non-toxic and which does not interact with any components of the composition in a deleterious manner.

“Sustained release” refers to the release of a therapeutically active compound and/or composition such that the blood levels of the therapeutically active compound are maintained within a desirable therapeutic range over an extended period of time. The sustained release formulation can be prepared using any conventional method known to one skilled in the art to obtain the desired release characteristics. Sustained release encompasses and includes extended release, delayed release, variable release, pulsed release, and the like.

“Hydralazine compound” refers to a compound having the formula:

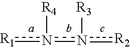

wherein a, b and c are each independently a single or a double bond; R₁ and R₂ are each independently a hydrogen, an alkyl, an ester or a heterocyclic ring; R₃ and R₄ are each independently a lone pair of electrons or a hydrogen, with the proviso that at least one of R₁, R₂, R₃ and R₄ is not a hydrogen. Exemplary hydralazine compounds include budralazine, cadralazine, dihydralazine, endralazine, hydralazine, pildralazine, todralazine and the like.

“Alkyl” refers to a lower alkyl group, a substituted lower alkyl group, a haloalkyl group, a hydroxyalkyl group, an alkenyl group, a substituted alkenyl group, an alkynyl group, a bridged cycloalkyl group, a cycloalkyl group or a heterocyclic ring, as defined herein. An alkyl group may also comprise one or more radical species, such as, for example a cycloalkylalkyl group or a heterocyclicalkyl group.

“Lower alkyl” refers to branched or straight chain acyclic alkyl group comprising one to about ten carbon atoms (preferably one to about eight carbon atoms, more preferably one to about six carbon atoms). Exemplary lower alkyl groups include methyl, ethyl, n-propyl, isopropyl, n-butyl, isobutyl, sec-butyl, t-butyl, pentyl, neopentyl, iso-amyl, hexyl, octyl, and the like.

“Substituted lower alkyl” refers to a lower alkyl group, as defined herein, wherein one or more of the hydrogen atoms have been replaced with one or more R¹⁰⁰ groups, wherein each R¹⁰⁰ is independently a hydroxy, an ester, an amidyl, an oxo, a carboxyl, a carboxamido, a halo, a cyano, a nitrate or an amino group, as defined herein.

“Haloalkyl” refers to a lower alkyl group, an alkenyl group, an alkynyl group, a bridged cycloalkyl group, a cycloalkyl group or a heterocyclic ring, as defined herein, to which is appended one or more halogens, as defined herein. Exemplary haloalkyl groups include trifluoromethyl, chloromethyl, 2-bromobutyl, 1-bromo-2-chloro-pentyl, and the like. “Alkenyl” refers to a branched or straight chain C₂-C₁₀ hydrocarbon (preferably a C₂-C₈ hydrocarbon, more preferably a C₂-C₆ hydrocarbon) that can comprise one or more carbon-carbon double bonds. Exemplary alkenyl groups include propylenyl, buten-1-yl, isobutenyl, penten-1-yl, 2,2-methylbuten-1-yl, 3-methylbuten-1-yl, hexan-1-yl, hepten-1-yl, octen-1-yl, and the like.

“Lower alkenyl” refers to a branched or straight chain C₂-C₄ hydrocarbon that can comprise one or two carbon-carbon double bonds.

“Substituted alkenyl” refers to a branched or straight chain C₂-C₁₀ hydrocarbon (preferably a C₂-C₈ hydrocarbon, more preferably a C₂-C₆ hydrocarbon) which can comprise one or more carbon-carbon double bonds, wherein one or more of the hydrogen atoms have been replaced with one or more R¹⁰⁰ groups, wherein each R¹⁰⁰ is independently a hydroxy, an oxo, a carboxyl, a carboxamido, a halo, a cyano or an amino group, as defined herein.

“Alkynyl” refers to an unsaturated acyclic C₂-C₁₀ hydrocarbon (preferably a C₂-C₈ hydrocarbon, more preferably a C₂-C₆ hydrocarbon) that can comprise one or more carbon-carbon triple bonds. Exemplary alkynyl groups include ethynyl, propynyl, butyn-1-yl, butyn-2-yl, pentyl-1-yl, pentyl-2-yl, 3-methylbutyn-1-yl, hexyl-1-yl, hexyl-2-yl, hexyl-3-yl, 3,3-dimethyl-butyn-1-yl, and the like.

“Bridged cycloalkyl” refers to two or more cycloalkyl groups, heterocyclic groups, or a combination thereof fused via adjacent or non-adjacent atoms. Bridged cycloalkyl groups can be unsubstituted or substituted with one, two or three substituents independently selected from alkyl, alkoxy, amino, alkylamino, dialkylamino, hydroxy, halo, carboxyl, alkylcarboxylic acid, aryl, amidyl, ester, alkylcarboxylic ester, carboxamido, alkylcarboxamido, oxo and nitro. Exemplary bridged cycloalkyl groups include adamantyl, decahydronapthyl, quinuclidyl, 2,6-dioxabicyclo(3.3.0)octane, 7-oxabicyclo(2.2.1)heptyl, 8-azabicyclo(3,2,1)oct-2-enyl and the like.

“Cycloalkyl” refers to a saturated or unsaturated cyclic hydrocarbon comprising from about 3 to about 10 carbon atoms. Cycloalkyl groups can be unsubstituted or substituted with one, two or three substituents independently selected from alkyl, alkoxy, amino, alkylamino, dialkylamino, arylamino, diarylamino, alkylarylamino, aryl, amidyl, ester, hydroxy, halo, carboxyl, alkylcarboxylic acid, alkylcarboxylic ester, carboxamido, alkylcarboxamido, oxo, alkylsulfinyl, and nitro. Exemplary cycloalkyl groups include cyclopropyl, cyclobutyl, cyclopentyl, cyclohexyl, cyclohexenyl, cyclohepta-1,3-dienyl, and the like.

“Heterocyclic ring or group” refers to a saturated or unsaturated cyclic hydrocarbon group having about 2 to about 10 carbon atoms (preferably about 4 to about 6 carbon atoms) where 1 to about 4 carbon atoms are replaced by one or more nitrogen, oxygen and/or sulfur atoms. Sulfur may be in the thio, sulfinyl or sulfonyl oxidation state. The heterocyclic ring or group can be fused to an aromatic hydrocarbon group. Heterocyclic groups can be unsubstituted or substituted with one, two or three substituents independently selected from alkyl, alkoxy, amino, alkylthio, aryloxy, arylthio, arylalkyl, hydroxy, oxo, thial, halo, carboxyl, carboxylic ester, alkylcarboxylic acid, alkylcarboxylic ester, aryl, arylcarboxylic acid, arylcarboxylic ester, amidyl, ester, alkylcarbonyl, arylcarbonyl, alkylsulfinyl, carboxamido, alkylcarboxamido, arylcarboxamido, sulfonic acid, sulfonic ester, sulfonamide nitrate and nitro. Exemplary heterocyclic groups include pyrrolyl, furyl, thienyl, 3-pyrrolinyl,4,5,6-trihydro-2H-pyranyl, pyridinyl, 1,4-dihydropyridinyl, pyrazolyl, triazolyl, pyrimidinyl, pyridazinyl, oxazolyl, thiazolyl, imidazolyl, indolyl, thiophenyl, furanyl, tetrahydrofuranyl, tetrazolyl, pyrrolinyl, pyrrolindinyl, oxazolindinyl 1,3-dioxolanyl, imidazolinyl, imidazolindinyl, pyrazolinyl, pyrazolidinyl, isoxazolyl, isothiazolyl, 1,2,3-oxadiazolyl, 1,2,3-triazolyl, 1,3,4-thiadiazolyl, 2H-pyranyl, 4H-pyranyl, piperidinyl, 1,4-dioxanyl, morpholinyl, 1,4-dithianyl, thiomorpholinyl, pyrazinyl, piperazinyl, 1,3,5-triazinyl, 1,3,5-trithianyl, benzo(b)thiophenyl, benzimidazolyl, benzothiazolinyl, quinolinyl, 2,6-dioxabicyclo(3.3.0)octane, and the like.

“Heterocyclic compounds” refer to mono- and polycyclic compounds comprising at least one aryl or heterocyclic ring.

“Aryl” refers to a monocyclic, bicyclic, carbocyclic or heterocyclic ring system comprising one or two aromatic rings. Exemplary aryl groups include phenyl, pyridyl, naphthyl, quinoyl, tetrahydronaphthyl, furanyl, indanyl, indenyl, indoyl, and the like. Aryl groups (including bicyclic aryl groups) can be unsubstituted or substituted with one, two or three substituents independently selected from alkyl, alkoxy, alkylthio, amino, alkylamino, dialkylamino, arylamino, diarylamino, alkylarylamino, halo, cyano, alkylsulfinyl, hydroxy, carboxyl, carboxylic ester, alkylcarboxylic acid, alkylcarboxylic ester, aryl, arylcarboxylic acid, arylcarboxylic ester, alkylcarbonyl, arylcarbonyl, amidyl, ester, carboxamido, alkylcarboxamido, carbomyl, sulfonic acid, sulfonic ester, sulfonamido and nitro. Exemplary substituted aryl groups include tetrafluorophenyl, pentafluorophenyl, sulfonamide, alkylsulfonyl, arylsulfonyl, and the like.

“Hydroxy” refers to —OH.

“Hydroxyalkyl” refers to a hydroxy group, as defined herein, appended to an alkyl group, as defined herein.

“Alkylcarbonyl” refers to R₅₂—C(O)—, wherein R₅₂ is an alkyl group, as defined herein.

“Arylcarbonyl” refers to R₅₅—C(O)—, wherein R₅₅ is an aryl group, as defined herein.

“Ester” refers to R₅₁C(O)O— wherein R₅₁ is a hydrogen atom, an alkyl group, an aryl group, an alkylaryl group, or an arylheterocyclic ring, as defined herein.

“Alkylaryl” refers to an alkyl group, as defined herein, to which is appended an aryl group, as defined herein. Exemplary alkylaryl groups include benzyl, phenylethyl, hydroxybenzyl, fluorobenzyl, fluorophenylethyl, and the like.

“Arylheterocyclic ring” refers to a bi- or tricyclic ring comprised of an aryl ring, as defined herein, appended via two adjacent carbon atoms of the aryl ring to a heterocyclic ring, as defined herein. Exemplary arylheterocyclic rings include dihydroindole, 1,2,3,4-tetra-hydroquinoline, and the like.

“Hydrazino” refers to H₂N—N(H)—.

In the invention, the preferred hydralazine compound is hydralazine, which is preferably administered in the form of a pharmaceutically acceptable salt and most preferably in the form of hydralazine hydrochloride. Hydralazine hydrochloride is commercially available from, for example, Lederle Standard Products, Pearl River, N.Y.; and Par Pharmaceuticals Inc., Spring Valley, N.Y. It is a white to off-white, crystalline powder and is soluble in water, slightly soluble in alcohol and very slightly soluble in ether.

Isosorbide dinitrate is commercially available, for example, under the trade names DILATRATE®-SR (Schwarz Pharma, Milwaukee, Wis.); ISORDIL® and ISORDILR TITRADOSE® (Wyeth Laboratories Inc., Philadelphia, Pa.); and SORBITRATE® (Zeneca Pharmaceuticals, Wilmington, Del.). Diluted isosorbide dinitrate (1,4,3,6-dianhydro-D-glucitol-2,5-dinitrate), USP, is a white to off-white powder. It is freely soluble in organic solvents such as ethanol, ether and chloroform, but is sparingly soluble in water.

Isosorbide mononitrate is commercially available, for example, under the trade names IMDUR® (A. B. Astra, Sweden); MONOKET® (Schwarz Pharma, Milwaukee, Wis.); and ISMO® (Wyeth-Ayerst Company, Philadelphia, Pa.).

The isosorbide dinitrate and isosorbide mononitrate can be stabilized to prevent explosions by the addition of compounds, such as, but not limited to, lactose, arginine, mannitol, sorbitol, cellulose (Avicel®) and the like, and combinations of two or more thereof.

The hydralazine compound and at least one of isosorbide dinitrate and isosorbide mononitrate can be administered as separate components or as components of the same composition. When the hydralazine compound and at least one of isosorbide dinitrate and isosorbide mononitrate are administered as separate components, they are preferably administered to the patient at about the same time. “About the same time” means that within about thirty minutes of administering one compound (e.g., the hydralazine compound or isosorbide dinitrate/mononitrate) to the patient, the other compound (e.g., isosorbide dinitrate/mononitrate or the hydralazine compound) is administered to the patient. “About the same time” also includes simultaneous administration of the compounds.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient a therapeutically effect amount of (i) a hydralazine compound (preferably hydralazine hydrochloride) and (ii) isosorbide dinitrate and/or isosorbide mononitrate (preferably isosorbide dinitrate). In one embodiment, the patient with heart failure has hypertension and/or endothelial dysfunction. In one embodiment, the patient is a black patient. In another embodiment, the patient with heart failure is categorized as New York Heart Association (NYHA) heart failure functional classification I, II, III or IV. The hydralazine compound (preferably hydralazine hydrochloride) and isosorbide dinitrate and/or isosorbide mononitrate (preferably isosorbide dinitrate) can be administered in the form of a composition or can be administered separately. In one embodiment, the patient is administered a therapeutically effective amount of hydralazine hydrochloride and isosorbide dinitrate. In another embodiment, the patient is administered hydralazine hydrochloride in an amount of about 30 milligrams to about 300 milligrams per day and isosorbide dinitrate in an amount of about 20 milligrams to about 200 milligrams per day. In another embodiment the patient is administered hydralazine hydrochloride in an amount of about 75 milligrams to about 225 milligrams per day and isosorbide dinitrate in an amount of about 40 milligrams to about 120 milligrams per day. In this embodiment the hydralazine may be administered as 75 mg once, twice or three times per day and the isosorbide dinitrate may be administered as 40 mg once, twice or three times per day. In another embodiment the patient is administered hydralazine hydrochloride in an amount of about 37.5 milligrams to about 112.5 milligrams per day and isosorbide dinitrate in an amount of about 20 milligrams to about 60 milligrams per day. In this embodiment the hydralazine may be administered as 37.5 mg once, twice or three times per day and the isosorbide dinitrate may be administered as 20 mg once, twice or three times per day. The hydralazine hydrochloride and isosorbide dinitrate can be administered separately or as components of the same composition.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient a therapeutically effective amount of (i) a hydralazine compound (preferably hydralazine hydrochloride), (ii) isosorbide dinitrate and/or isosorbide mononitrate (preferably isosorbide dinitrate), and (iii) optionally at least one compound selected from the group consisting of an angiotensin converting enzyme inhibitor, a β-adrenergic antagonist, an angiotensin II antagonist, an aldosterone antagonist, a cardiac glycoside, a diuretic compound, a non-steroidal anti-inflammatory compound or a combination of two or more thereof. In another embodiment, the invention provides methods of administering (i) a hydralazine compound (preferably hydralazine hydrochloride), (ii) isosorbide dinitrate and/or isosorbide mononitrate (preferably isosorbide dinitrate), and (iii) an angiotensin converting enzyme inhibitor. In another embodiment, the invention provides methods of administering (i) a hydralazine compound (preferably hydralazine hydrochloride), (ii) isosorbide dinitrate and/or isosorbide mononitrate (preferably isosorbide dinitrate), and (iii) a β-adrenergic antagonist. In another embodiment, the invention provides methods of administering (i) a hydralazine compound (preferably hydralazine hydrochloride), (ii) isosorbide dinitrate and/or isosorbide mononitrate (preferably isosorbide dinitrate), and (iii) an angiotensin II antagonist. In another embodiment, the invention provides methods of administering (i) a hydralazine compound (preferably hydralazine hydrochloride), (ii) isosorbide dinitrate and/or isosorbide mononitrate (preferably isosorbide dinitrate), and (iii) an aldosterone antagonist. In another embodiment, the invention provides methods of administering (i) a hydralazine compound (preferably hydralazine hydrochloride), (ii) isosorbide dinitrate and/or isosorbide mononitrate (preferably isosorbide dinitrate), and (iii) a cardiac glycoside. In another embodiment, the invention provides methods of administering (i) a hydralazine compound (preferably hydralazine hydrochloride), (ii) isosorbide dinitrate and/or isosorbide mononitrate (preferably isosorbide dinitrate), and (iii) a diuretic compound. In another embodiment, the invention provides methods of administering (i) a hydralazine compound (preferably hydralazine hydrochloride), (ii) isosorbide dinitrate and/or isosorbide mononitrate (preferably isosorbide dinitrate), (iii) an angiotensin converting enzyme inhibitor, and (iv) a β-adrenergic antagonist. In another embodiment, the invention provides methods of administering (i) a hydralazine compound (preferably hydralazine hydrochloride), (ii) isosorbide dinitrate and/or isosorbide mononitrate (preferably isosorbide dinitrate), (iii) an angiotensin converting enzyme inhibitor, and (iv) an aldosterone antagonist. In another embodiment, the invention provides methods of administering (i) a hydralazine compound (preferably hydralazine hydrochloride), (ii) isosorbide dinitrate and/or isosorbide mononitrate (preferably isosorbide dinitrate), (iii) an angiotensin converting enzyme inhibitor, and (iv) an angiotensin II antagonist. In another embodiment, the invention provides methods of administering (i) a hydralazine compound (preferably hydralazine hydrochloride), (ii) isosorbide dinitrate and/or isosorbide mononitrate (preferably isosorbide dinitrate), (iii) a β-adrenergic antagonist, and (iv) an aldosterone antagonist. In another embodiment, the invention provides methods of administering (i) a hydralazine compound (preferably hydralazine hydrochloride), (ii) isosorbide dinitrate and/or isosorbide mononitrate (preferably isosorbide dinitrate), (iii) a β-adrenergic antagonist, and (iv) an angiotensin II antagonist. In another embodiment, the invention provides methods of administering (i) a hydralazine compound (preferably hydralazine hydrochloride), (ii) isosorbide dinitrate and/or isosorbide mononitrate (preferably isosorbide dinitrate), (iii) an angiotensin converting enzyme inhibitor, (iv) a β-adrenergic antagonist, and (v) an aldosterone antagonist. In another embodiment, the invention provides methods of administering (i) a hydralazine compound (preferably hydralazine hydrochloride), (ii) isosorbide dinitrate and/or isosorbide mononitrate (preferably isosorbide dinitrate), (iii) an angiotensin converting enzyme inhibitor, (iv) a β-adrenergic antagonist, and (v) an angiotensin II antagonist. In another embodiment, the invention provides methods of administering (i) a hydralazine compound (preferably hydralazine hydrochloride), (ii) isosorbide dinitrate and/or isosorbide mononitrate (preferably isosorbide dinitrate), (iii) an angiotensin II antagonist and (iv) an aldosterone antagonist. In another embodiment, the invention provides methods of administering (i) a hydralazine compound (preferably hydralazine hydrochloride), (ii) isosorbide dinitrate and/or isosorbide mononitrate (preferably isosorbide dinitrate), (iii) a diuretic compound, and (iv) a cardiac glycoside. In another embodiment, the invention provides methods of administering (i) a hydralazine compound (preferably hydralazine hydrochloride), (ii) isosorbide dinitrate and/or isosorbide mononitrate (preferably isosorbide dinitrate), and (iii) a non-steroidal anti-inflammatory compound. In another embodiment, the patient is black. In another embodiment, the patient with heart failure is categorized as New York Heart Association (NYHA) heart failure functional classification I, II, III or IV. In these embodiments the hydralazine compound, and at least one of isosorbide dinitrate and isosorbide mononitrate can be administered separately or as components of the same composition, and can be administered in the form of a composition with or simultaneously with, subsequently to, or prior to administration of at least one of the angiotensin converting enzyme inhibitor, β-adrenergic antagonist, angiotensin II antagonist, aldosterone antagonist, cardiac glycoside, diuretic compound, non-steroidal anti-inflammatory compound or combinations of two or more thereof. In one embodiment, all the compounds are administered together in the form of a single composition.

In another embodiment, the invention provides pharmaceutical kits comprising at least one hydralazine compound or a pharmaceutically acceptable salt thereof, at least one of isosorbide dinitrate and isosorbide mononitrate, and, optionally, at least one of an angiotensin converting enzyme inhibitor, a β-adrenergic antagonist, an angiotensin II antagonist, an aldosterone antagonist, a cardiac glycosides, a diuretic compound, a non-steroidal anti-inflammatory compound, and combinations of two or more thereof. The hydralazine compound or a pharmaceutically acceptable salt thereof, and at least one of isosorbide dinitrate and isosorbide mononitrate, and, optionally, an angiotensin converting enzyme inhibitor, a β-adrenergic antagonist, an angiotensin II antagonist, an aldosterone antagonist, a cardiac glycoside and a diuretic compound, can be separate components in the kit or can be in the form of a composition in the kit in one or more pharmaceutically acceptable carriers.

In one embodiment, the hydralazine hydrochloride can be administered in an amount of about 30 milligrams per day to about 400 milligrams per day; the isosorbide dinitrate can be administered in an amount of about 10 milligrams per day to about 200 milligrams per day; or the isosorbide mononitrate can be administered in an amount of about 5 milligrams per day to about 120 milligrams per day. In another embodiment, the hydralazine hydrochloride can be administered in an amount of about 50 milligrams per day to about 300 milligrams per day; the isosorbide dinitrate can be administered in an amount of about 20 milligrams per day to about 160 milligrams per day; or the isosorbide mononitrate can be administered in an amount of about 15 milligrams per day to about 100 milligrams per day. In another embodiment, the hydralazine hydrochloride can be administered in an amount of about 37.5 milligrams to about 75 milligrams one to four times per day; the isosorbide dinitrate can be administered in an amount of about 20 milligrams to about 40 milligrams one to four times per day; or the isosorbide mononitrate can be administered in an amount of about 10 milligrams to about 20 milligrams one to four times per day. The particular amounts of hydralazine and isosorbide dinitrate or isosorbide mononitrate can be administered as a single dose once a day; or in multiple doses several times throughout the day; or as a sustained-release oral formulation.

In one embodiment of the methods of the invention, the patient can be administered a composition comprising about 225 mg hydralazine hydrochloride and about 120 mg isosorbide dinitrate once per day (i.e., q.d.). In another embodiment of the methods of the invention, the patient can be administered a composition comprising about 112.5 mg hydralazine hydrochloride and about 60 mg isosorbide dinitrate twice per day (i.e., b.i.d.). In another embodiment of the methods of the invention, the patient can be administered a composition comprising about 56.25 mg hydralazine hydrochloride and about 30 mg isosorbide dinitrate twice per day (i.e., b.i.d.). In another embodiment of the methods of the invention, the patient can be administered a composition comprising about 75 mg hydralazine hydrochloride and about 40 mg isosorbide dinitrate three times per day (i.e., t.i.d.). In another embodiment of the methods of the invention, the patient can be administered a composition comprising about 37.5 mg hydralazine hydrochloride and about 20 mg isosorbide dinitrate three times per day (i.e., t.i.d.).

In any of the embodiments described herein, the patient can be administered one, two or three compositions (e.g., two tablets, two capsules and the like) at any particular time. For example, the patient can be administered two separate compositions, wherein each composition comprises about 112.5 mg hydralazine hydrochloride and about 60 mg isosorbide dinitrate twice per day (i.e., b.i.d.). In another embodiment, the patient can be administered two separate compositions, wherein each composition comprises about 56.25 mg hydralazine hydrochloride and about 30 mg isosorbide dinitrate twice per day (i.e., b.i.d.).

In the invention the at least one hydralazine compound or pharmaceutically acceptable salts thereof, and at least one of isosorbide dinitrate and isosorbide mononitrate, are administered as separate components or as components of the same composition with at least one of the angiotensin converting enzyme inhibitor, β-adrenergic antagonist, angiotensin II antagonist, aldosterone antagonist, cardiac glycoside, diuretic compound, non-steroidal antiinflammatory compound or a combination of two or more thereof. They can also be administered as separate components as single doses once a day; or in multiple doses several times throughout the day; or as a sustained-release oral formulation.

In one embodiment, the invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient a therapeutically effective amount of (i) at least one hydralazine compound or a pharmaceutically acceptable salt thereof (e.g., preferably hydralazine hydrochloride), (ii) at least one of isosorbide dinitrate and isosorbide mononitrate (e.g., preferably isosorbide dinitrate), and (iii) optionally an angiotensin-converting enzyme inhibitor. Suitable angiotensin-converting enzyme inhibitors (ACE inhibitors) include, but are not limited to, alacepril, benazepril (LOTENSIN®, CIBACEN®), benazeprilat, captopril, ceronapril, cilazapril, delapril, duinapril, enalapril, enalaprilat, fasidotril, fosinopril, fosinoprilat, gemopatrilat, glycopril, idrapril, imidapril, lisinopril, moexipril, moveltipril, naphthopidil, omapatrilat, pentopril, perindopril, perindoprilat, quinapril, quinaprilat, ramipril, ramiprilat, rentipril, saralasin acetate, spirapril, temocapril, trandolapril, trandolaprilat, urapidil, zofenopril, acylmercapto and mercaptoalkanoyl pralines, carboxyalkyl dipeptides, carboxyalkyl dipeptide, phosphinylalkanoyl pralines, registry no. 796406, AVE 7688, BP1.137, CHF 1514, E 4030, ER 3295, FPL-66564, MDL 100240, RL 6134, RL 6207, RL 6893, SA 760, S-5590, Z 13752A, and the like. One skilled in the art will appreciate that the angiotensin-converting enzyme inhibitors may be administered in the form of pharmaceutically acceptable salts, hydrates, acids and/or stereoisomers thereof. Suitable angiotensin-converting enzyme inhibitors are described more fully in the literature, such as in Goodman and Gilman, The Pharmacological Basis of Therapeutics (9th Edition), McGraw-Hill, 1995; and the Merck Index on CD-ROM, Twelfth Edition, Version 12:1, 1996; and on STN Express, file phar and file registry.

In some embodiments the angiotensin-converting enzyme inhibitors are benazepril, captopril, enalapril, fosinopril, lisinopril, moexipril, quinapril, ramipril, trandolapril or trandolaprilat. In other embodiments the benazepril is administered as benazepril hydrochloride in an amount of about 5 milligrams to about 80 milligrams as a single dose or as multiple doses per day; the captopril is administered in an amount of about 12.5 milligrams to about 450 milligrams as a single dose or as multiple doses per day; the enalapril is administered as enalapril maleate in an amount of about 2.5 milligrams to about 40 milligrams as a single dose or as multiple doses per day; the fosinopril is administered as fosinopril sodium in an amount of about 5 milligrams to about 60 milligrams as a single dose or as multiple doses per day; the lisinopril is administered in an amount of about 2.5 milligrams to about 75 milligrams as a single dose or as multiple doses per day; the moexipril is administered as moexipril hydrochloride in an amount of about 7.5 milligrams to about 45 milligrams as a single dose or as multiple doses per day; the quinapril is administered as quinapril hydrochloride in an amount of about 5 milligrams to about 40 milligrams as single or multiple doses per day; the ramapril hydrochloride is administered in an amount of about 1.25 milligrams to about 40 milligrams as single or multiple doses per day; the trandolapril is administered in an amount of about 0.5 milligrams to about 4 milligrams as single or multiple doses per day; the trandolaprilat is administered in an amount of about 0.5 milligrams to about 4 milligrams as single or multiple doses per day. In other embodiments the angiotensin-converting enzyme inhibitors are captopril, enalapril or lisinopril.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient three times per day (i) about 37.5 milligrams to about 75 milligrams hydralazine hydrochloride, about 20 milligrams to about 40 milligrams isosorbide dinitrate and about 12.5 milligrams captopril; (ii) about 37.5 milligrams to about 75 milligrams hydralazine hydrochloride, about 20 milligrams to about 40 milligrams isosorbide dinitrate and about 25 milligrams captopril; (iii) about 37.5 milligrams to about 75 milligrams hydralazine hydrochloride, about 20 milligrams to about 40 milligrams isosorbide dinitrate and about 50 milligrams captopril; or (iv) about 37.5 milligrams to about 75 milligrams hydralazine hydrochloride, about 20 milligrams to about 40 milligrams isosorbide dinitrate and about 100 milligrams captopril. In these embodiments the hydralazine hydrochloride, isosorbide dinitrate and captopril can be administered separately or in the form of a composition.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient twice per day (i) about 56.25 milligrams to about 112.5 milligrams hydralazine hydrochloride, about 30 milligrams to about 60 milligrams isosorbide dinitrate and about 2.5 milligrams enalapril; (ii) about 56.25 milligrams to about 112.5 milligrams hydralazine hydrochloride, about 30 milligrams to about 60 milligrams isosorbide dinitrate and about 5 milligrams enalapril; (iii) about 56.25 milligrams to about 112.5 milligrams hydralazine hydrochloride, about 30 milligrams to about 60 milligrams isosorbide dinitrate and about 10 milligrams enalapril; or (iv) about 56.25 milligrams to about 112.5 milligrams hydralazine hydrochloride, about 30 milligrams to about 60 milligrams isosorbide dinitrate and about 20 milligrams enalapril. In these embodiments the hydralazine hydrochloride, isosorbide dinitrate and enalapril can be administered separately or in the form of a composition.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient need thereof comprising administering to the patient once per day (i) about 112.5 milligrams to about 225 milligrams hydralazine hydrochloride, about 60 milligrams to about 120 milligrams isosorbide dinitrate and about 2.5 milligrams lisinopril; (ii) about 112.5 milligrams to about 225 milligrams hydralazine hydrochloride, about 60 milligrams to about 120 milligrams isosorbide dinitrate and about 5 milligrams lisinopril; (iii) about 112.5 milligrams to about 225 milligrams hydralazine hydrochloride, about 60 milligrams to about 120 milligrams isosorbide dinitrate and about 10 milligrams lisinopril; or (iv) about 112.5 milligrams to about 225 milligrams hydralazine hydrochloride, about 60 milligrams to about 120 milligrams isosorbide dinitrate and about 20 milligrams lisinopril. In these embodiments the hydralazine hydrochloride, isosorbide dinitrate and lisinopril can be administered separately or in the form of a composition.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient a therapeutically effective amount of (i) at least one hydralazine compound or a pharmaceutically acceptable salt thereof (e.g., preferably hydralazine hydrochloride), (ii) at least one of isosorbide dinitrate and isosorbide mononitrate (e.g., preferably isosorbide dinitrate), and (iii) a β-adrenergic antagonist. Suitable β-adrenergic antagonists include, but are not limited to, acebutolol, alprenolol, amosulalol, arotinolol, atenolol, befunolol, betaxolol, bevantolol, bisoprolol, bopindolol, bucindolol, bucumolol, bufetolol, bufuralol, bunitrolol, bupranolol, butofilolol, carazolol, capsinolol, carteolol, carvedilol (COREG®), celiprolol, cetamolol, cindolol, cloranolol, dilevalol, diprafenone, epanolol, ersentilide, esmolol, esprolol, hydroxalol, indenolol, labetalol, landiolol, laniolol, levobunolol, mepindolol, methylpranol, metindol, metipranolol, metrizoranolol, metoprolol, moprolol, nadolol, nadoxolol, nebivolol, nifenalol, nipradilol, oxprenolol, penbutolol, pindolol, practolol, pronethalol, propranolol, sotalol, sotalolnadolol, sulfinalol, taliprolol, talinolol, tertatolol, tilisolol, timolol, toliprolol, tomalolol, trimepranol, xamoterol, xibenolol, 2-(3-(1,1-dimethylethyl)-amino-2-hydroxypropoxy)-3-pyridenecarbonitrilHCl, 1-butylamino-3-(2,5-dichlorophenoxy)-2-propanol, 1-isopropylamino-3-(4-(2-cyclopropylmethoxyethyl) phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol, 7-(2-hydroxy-3-t-butylaminpropoxy)phthalide, Acc 9369, AMO-140, BIB-16S, CP-331684, Fr-172516, ISV-208, L-653328, LM-2616, SB-226552, SR-58894A, SR-59230A, TZC-5665, UK-1745, YM-430, and the like. One skilled in the art will appreciate that the β-adrenergic antagonists can be administered in the form of pharmaceutically acceptable salts and/or stereoisomers. Suitable β-adrenergic antagonists are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

In some embodiments the β-adrenergic antagonists are atenolol, bisoprolol, carvedilol, metoprolol, nebivolol, propranolol or timolol. In other embodiments the atenolol is administered in an amount of about 50 milligrams to about 200 milligrams as a single dose or as multiple doses per day; the bisoprolol is administered as bisoprolol fumarate in an amount of about 2.5 milligrams to about 30 milligrams as a single dose or as multiple doses per day; the carvedilol is administered in an amount of about 3.125 milligrams to about 200 milligrams as a single dose or as multiple doses per day; the metoprolol is administered as metoprolol tartarate or metoprolol succinate in an amount of about 25 milligrams to about 300 milligrams as a single dose or as multiple doses per day; the nebivolol is administered as nebivolol hydrochloride in an amount of about 2.5 milligrams to about 20 milligrams as a single dose or as multiple doses per day; the propranolol is administered as propranolol hydrochloride in an amount of about 40 milligrams to about 240 milligrams as a single dose or as multiple doses per day; the timolol is administered as timolol maleate in an amount of about 10 milligrams to about 30 milligrams as a single dose or as multiple doses per day.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient twice per day (i) about 56.25 milligrams to about 112.5 milligrams hydralazine hydrochloride, about 30 milligrams to about 60 milligrams isosorbide dinitrate and about 3.125 milligrams carvedilol; (ii) about 56.25 milligrams to about 112.5 milligrams hydralazine hydrochloride, about 30 milligrams to about 60 milligrams isosorbide dinitrate and about 6.25 milligrams carvedilol; (iii) about 56.25 milligrams to about 112.5 milligrams hydralazine hydrochloride, about 30 milligrams to about 60 milligrams isosorbide dinitrate and about 12.5 milligrams carvedilol; or (iv) about 56.25 milligrams to about 112.5 milligrams hydralazine hydrochloride, about 30 milligrams to about 60 milligrams isosorbide dinitrate and about 25 milligrams carvedilol. In these embodiments the hydralazine hydrochloride, isosorbide dinitrate and carvedilol can be administered separately or in the form of a composition.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient once per day (i) about 112.5 milligrams to about 225 milligrams hydralazine hydrochloride, about 60 milligrams to about 120 milligrams isosorbide dinitrate and about 25 milligrams metoprolol; (ii) about 112.5 milligrams to about 225 milligrams hydralazine hydrochloride, about 60 milligrams to about 120 milligrams isosorbide dinitrate and about 50 milligrams metoprolol; (iii) about 112.5 milligrams to about 225 milligrams hydralazine hydrochloride, about 60 milligrams to about 120 milligrams isosorbide dinitrate and about 100 milligrams metoprolol; or (iv) about 112.5 milligrams to about 225 milligrams hydralazine hydrochloride, about 60 milligrams to about 120 milligrams isosorbide dinitrate and about 200 milligrams metoprolol. In these embodiments the hydralazine hydrochloride, isosorbide dinitrate and metoprolol can be administered separately or in the form of a composition.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient once per day (i) about 112.5 milligrams to about 225 milligrams hydralazine hydrochloride, about 60 milligrams to about 120 milligrams isosorbide dinitrate and about 2.5 milligrams nebivolol; (ii) about 112.5 milligrams to about 225 milligrams hydralazine hydrochloride, about 60 milligrams to about 120 milligrams isosorbide dinitrate and about 5 milligrams nebivolol; (iii) about 112.5 milligrams to about 225 milligrams hydralazine hydrochloride, about 60 milligrams to about 120 milligrams isosorbide dinitrate and about 10 milligrams nebivolol; or (iv) about 112.5 milligrams to about 225 milligrams hydralazine hydrochloride, about 60 milligrams to about 120 milligrams isosorbide dinitrate and about 20 milligrams nebivolol. In these embodiments the hydralazine hydrochloride, isosorbide dinitrate and nebivolol can be administered separately or in the form of a composition.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient a therapeutically effective amount of (i) at least one hydralazine compound or a pharmaceutically acceptable salt thereof (e.g., preferably hydralazine hydrochloride), (ii) at least one of isosorbide dinitrate and isosorbide mononitrate (e.g., preferably isosorbide dinitrate), and (iii) an angiotensin II antagonist Suitable angiotensin II antagonists include, but are not limited to, angiotensin, abitesartan, candesartan, candesartan cilexetil, elisartan, embusartan, enoltasosartan, eprosartan, fonsartan, forasartan, glycyllosartan, irbesartan, losartan, olmesartan, milfasartan, medoxomil, ripisartan, pratosartan, saprisartan, saralasin, sarmesin, tasosartan, telmisartan, valsartan, zolasartan, 3-(2′(tetrazole-5-yl)-1,1′-biphen-4-yl)methyl-5,7-dimethyl-2-ethyl-3H-imidazo[4,5-b]pyridine, antibodies to angiotensin II, A-81282, A-81988, BAY 106734, BIBR-363, BIBS-39, BIBS-222, BMS-180560, BMS-184698, BMS-346567, CGP-38560A, CGP-42112A, CGP-48369, CGP-49870, CGP-63170, CI-996, CP-148130, CL-329167, CV-11194, DA-2079, DE-3489, DMP-811, DuP-167, DuP-532, DuP-753, E-1477, E-4177, E-4188, EMD-66397, EMD-666R4, EMD-73495, EMD-66684, EXP-063, EXP-929, EXP-3174, EXP-6155, EXP-6803, EXP-7711, EXP-9270, EXP-9954, FK-739, FRI 153332, GA-0050, GA-0056, HN-65021, HOE-720, HR-720, ICI-D6888, ICI-D7155, ICI-D8731, KRI-1177, KT3-671, KT-3579, KW-3433, L-158809, L-158978, L-159282, L-159689, L-159874, L-161177, L-162154, L-162234, L-162441, L-163007, L-163017, LF-70156, LRB-057, LRB-081, LRB-087, LY-235656, LY-266099, LY-285434, LY-301875, LY-302289, LY-315995, ME-3221, MK-954, PD-123177, PD-123319, PD-126055, PD-150304, RG-13647, RWJ-38970, RWJ-46458, S-8307, S-8308, SC-51757, SC-54629, SC-52458, SC-52459, SK 1080, SL-910102, SR-47436, TAK-536, UP-2696, U-96849, U-97018, UK-77778, UP-275-22, WAY-126227, WK-1260, WK-1360, WK-1492, WY 126227, YH-1498, YM-358, YM-31472, X-6803, XH-148, XR-510, ZD-6888, ZD-7155, ZD-8731, ZD 8131, the compounds of ACS registry numbers 124750-92-1, 133240-46-7, 135070-05-2, 139958-16-0, 145160-84-5, 147403-03-0, 153806-29-2, 439904-54-8P, 439904-55-9P, 439904-56-0P, 439904-57-1P, 439904-58-2P, 155918-60-8P, 155918-61-9P, 272438-16-1P, 272446-75-0P, 223926-77-0P, 169281-89-4, 439904-65-1P, 165113-01-9P, 165113-02-0P, 165113-03-1P, 165113-03-2P, 165113-05-3P, 165113-06-4P, 165113-07-5P, 165113-08-6P, 165113-09-7P, 165113-10-0P, 165113-11-1P, 165113-12-2P, 165113-17-7P, 165113-18-8P, 165113-19-9P, 165113-20-2P, 165113-13-3P, 165113-14-4P, 165113-15-5P, 165113-16-6P, 165113-21-3P, 165113-22-4P, 165113-23-5P, 165113-24-6P, 165113-25-7P, 165113-26-8P, 165113-27-9P, 165113-28-0P, 165113-29-1P, 165113-30-4P, 165113-31-5P, 165113-32-6P, 165113-33-7P, 165113-34-8P, 165113-35-9P, 165113-36-0P, 165113-37-1P, 165113-38-2P, 165113-39-3P, 165113-40-6P, 165113-41-7P, 165113-42-8P, 165113-43-9P, 165113-44-0P, 165113-45-1P, 165113-46-2P, 165113-47-3P, 165113-48-4P, 165113-49-5P, 165113-50-8P, 165113-51-9P, 165113-52-0P, 165113-53-1P, 165113-54-2P, 165113-55-3P, 165113-56-4P, 165113-57-5P, 165113-58-6P, 165113-59-7P, 165113-60-0P, 165113-61-1P, 165113-62-2P, 165113-63-3P, 165113-64-4P, 165113-65-5P, 165113-66-6P, 165113-67-7P, 165113-68-8P, 165113-69-9P, 165113-70-2P, 165113-71-3P, 165113-72-4P, 165113-73-5P, 165113-74-6P, 114798-27-5, 114798-28-6, 114798-29-7, 124749-82-2, 114798-28-6, 124749-84-4, 124750-88-5, 124750-91-0, 124750-93-2, 161946-65-2P, 161947-47-3P, 161947-48-4P, 161947-51-9P, 161947-52-0P, 161947-55-3P, 161947-56-4P, 161947-60-0P, 161947-61-1P, 161947-68-8P, 161947-69-9P, 161947-70-2P, 161947-71-3P, 161947-72-4P, 161947-74-6P, 161947-75-7P, 161947-81-5P, 161947-82-6P, 161947-83-7P, 161947-84-8P, 161947-85-9P, 161947-86-0P, 161947-87-1P, 161947-88-2P, 161947-89-3P, 161947-90-6P, 161947-91-7P, 161947-92-8P, 161947-93-9P, 161947-94-0P, 161947-95-1P, 161947-96-2P, 161947-97-3P, 161947-98-4P, 161947-99-5P, 161948-00-1P, 161948-01-2P, 161948-02-3P, 168686-32-6P, 167301-42-0P, 166813-82-7P, 166961-56-4P, 166961-58-6P, 158872-96-9P, 158872-97-0P, 158807-14-8P, 158807-15-9P, 158807-16-0P, 158807-17-1P, 158807-18-2P, 158807-19-3P, 158807-20-6P, 155884-08-5P, 154749-99-2, 167371-59-7P, 244126-99-6P, 177848-35-0P and 141309-82-2P, and the like. One skilled in the art will appreciate that the angiotensin II antagonists can be administered in the form of pharmaceutically acceptable salts and/or stereoisomers. Suitable angiotensin II antagonists are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

In one embodiment the angiotensin II antagonists are candesartan, eprosartan, irbesartan, losartan, omlesartan, telmisartan or valsartan. In other embodiments the candesartan is administered as candesartan cilexetil in an amount of about 15 milligrams to about 100 milligrams as a single dose or as multiple doses per day; the eprosartan is administered as eprosartan mesylate in an amount of about 400 milligrams to about 1600 milligrams as a single dose or as multiple doses per day; the irbesartan is administered in an amount of about 75 milligrams to about 1200 milligrams as a single dose or as multiple doses per day; the losartan is administered as losartan potassium in an amount of about 25 milligrams to about 100 milligrams as a single dose or as multiple doses per day; the omlesartan is administered as omlesartan medoxomil in an amount of about 5 milligrams to about 40 milligrams as a single dose or as multiple doses per day; the telmisartan is administered in an amount of about 20 milligrams to about 80 milligrams as a single dose or as multiple doses per day; the valsartan is administered in an amount of about 80 milligrams to about 320 milligrams as a single dose or as multiple doses per day.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient a therapeutically effective amount of (i) at least one hydralazine compound or a pharmaceutically acceptable salt thereof (e.g., preferably hydralazine hydrochloride), (ii) at least one of isosorbide dinitrate and isosorbide mononitrate (e.g., preferably isosorbide dinitrate), and (iii) an aldosterone antagonist. Suitable aldosterone antagonists include, but are not limited to, canrenone, potassium canrenoate, drospirenone, spironolactone, eplerenone (INSPRA®), epoxymexrenone, fadrozole, pregn-4-ene-7,2,1-dicarboxylic acid, 9,11-epoxy-17-hydroxy-3-oxo, γ-lactone, methyl ester, (7α,11α,17β.)-; pregn-4-ene-7,21-dicarboxylic acid, 9,11-epoxy-17-hydroxy-3-oxo-dimethyl ester, (7α,11α,17β.)-; 3′H-cyclopropa(6,7)pregna-4,6-diene-21-carboxylic acid, 9,11-epoxy-6,7-dihydro-17-hydroxy-3-oxo-, γ-lactone, (6β,7β,11α,17β)-; pregn-4-ene-7,2,1-dicarboxylic acid, 9,11-epoxy-17-hydroxy-3-oxo-, 7-(1-methylethyl) ester, monopotassium salt, (7α,11α,17β.)-; pregn-4-ene-7,21-dicarboxylic acid, 9,11-epoxy-17-hydroxy-3-oxo-, 7-methyl ester, monopotassium salt, (7α,11α,17β.)-; 3′H-cyclopropa(6,7) pregna-1,4,6-triene-21-carboxylic acid, 9,11-epoxy-6,7-dihydro-17-hydroxy-3-oxo-, γ-lactone, (6β,7β,11α)-; 3′H-cyclopropa(6,7)pregna-4,6-diene-21-carboxylic acid, 9,11-epoxy-6,7-dihydro-17-hydroxy-3-oxo-, methyl ester, (6β,7β,11α,17β)-; 3′H-cyclopropa (6,7)pregna-4,6-diene-21-carboxylic acid, 9,11-epoxy-6,7-dihydro-17-hydroxy-3-oxo-, monopotassium salt, (6β,7β,11α,17β)-; 3′H-cyclopropa(6,7)pregna-1,4,6-triene-21-carboxylic acid, 9,11-epoxy-6,7-dihydro-17-hydroxy-3-oxo-, γ-lactone, (6β,7β,11α,17β)-; pregn-4-ene-7,2,1-dicarboxylic acid, 9,11-epoxy-17-hydroxy-3-oxo-, γ-lactone, ethyl ester, (7α,11α,17β)-; pregn-4-ene-7,21-dicarboxylic acid, 9,11-epoxy-17-hydroxy-3-oxo-, γ-lactone, 1-methylethyl ester, (7α,11α,17β)-; RU-28318, and the like. One skilled in the art will appreciate that the aldosterone antagonists can be administered in the form of their pharmaceutically acceptable salts and/or stereoisomers. Suitable aldosterone antagonists are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

In some embodiments, the aldosterone antagonist is eplerenone or spironolactone (a potassium sparing diuretic that acts like an aldosterone antagonist). In one embodiment eplerenone is administered in an amount of about 25 milligrams to about 300 milligrams as a single dose or as multiple doses per day; the spironolactone is administered in an amount of about 25 milligrams to about 150 milligrams as a single dose or as multiple doses per day.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient a therapeutically effective amount of (i) at least one hydralazine compound or a pharmaceutically acceptable salt thereof (e.g., preferably hydralazine hydrochloride), (ii) at least one of isosorbide dinitrate and isosorbide mononitrate (e.g., preferably isosorbide dinitrate), and (iii) spironolactone. The compounds can be administered separately or in the form of a composition.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient a therapeutically effective amount of (i) at least one hydralazine compound or a pharmaceutically acceptable salt thereof (e.g., preferably hydralazine hydrochloride), (ii) at least one of isosorbide dinitrate and isosorbide mononitrate (e.g., preferably isosorbide dinitrate), and (iii) eplerenone. The compounds can be administered separately or in the form of a composition.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient a therapeutically effective amount of (i) at least one hydralazine compound or a pharmaceutically acceptable salt thereof (e.g., preferably hydralazine hydrochloride), (ii) at least one of isosorbide dinitrate and isosorbide mononitrate (e.g., preferably isosorbide dinitrate), and (iii) one or more diuretics. Suitable diuretics include but are not limited to, thiazides (such as, for example, althiazide, bendroflumethiazide, benzclortriazide, benzhydrochlorothiazide, benzthiazide, buthiazide, chlorothiazide, cyclopenethiazide, cyclothiazide, epithiazide, ethiazide, hydrobenzthiazide, hydrochlorothiazide, hydroflumethiazide, methylclothiazide, methylcyclothiazide, penflutazide, polythiazide, teclothiazide, trichlormethiazide, triflumethazide, and the like); alilusem, ambuside, amiloride, aminometradine, azosemide, bemetizide, bumetanide, butazolamide, butizide, canrenone, carperitide, chloraminophenamide, chlorazanil, chlormerodrin, chlorthalidone, cicletanide, clofenamide, clopamide, clorexolone, conivaptan, daglutril, dichlorophenamide, disulfamide, ethacrynic acid, ethoxzolamide, etozolon, fenoldopam, fenquizone, furosemide, indapamide, mebutizide, mefruside, meralluride, mercaptomerin sodium, mercumallylic acid, mersalyl, methazolamide, meticane, metolazone, mozavaptan, muzolimine, N-(5-1,3,4-thiadiazol-2-yl)acetamide, nesiritide, pamabrom, paraflutizide, piretanide, protheobromine, quinethazone, scoparius, spironolactone, theobromine, ticrynafen, torsemide, torvaptan, triamterene, tripamide, ularitide, xipamide or potassium, AT 189000, AY 31906, BG 9928, BG 9791, C 2921, DTI 0017, JDL 961, KW 3902, MCC 134, SLV 306, SR 121463, WAY 140288, ZP 120, and the like. One skilled in the art will appreciate that the diuretics can be administered in the form of their pharmaceutically acceptable salts and/or stereoisomers. Suitable diuretics are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Depending on the diuretic employed, potassium may also be administered to the patient in order to optimize the fluid balance while avoiding hypokalemic alkalosis. The administration of potassium can be in the form of potassium chloride or by the daily ingestion of foods with high potassium content such as, for example, bananas or orange juice. The method of administration of these compounds is described in further detail in U.S. Pat. No. 4,868,179, the disclosure of which is incorporated by reference herein in its entirety.

In some embodiments, the diuretics are amiloride, furosemide, chlorthalidone, chlorothiazide, hydrochlorothiazide, hydroflumethiazide, or triamterene. In other embodiments the amiloride is administered as amiloride hydrochloride in an amount of about 5 milligrams to about 15 milligrams as a single dose or as multiple doses per day; the furosemide is administered in an amount of about 10 milligrams to about 600 milligrams as a single dose or as multiple doses per day; the chlorthalidone is administered in an amount of about 15 milligrams to about 150 milligrams as a single dose or as multiple doses per day; the chlorothiazide is administered in an amount of about 500 milligrams to about 2 grams as a single dose or as multiple doses per day; the hydrochlorothiazide is administered in an amount of about 12.5 milligrams to about 300 milligrams as a single dose or as multiple doses per day; the hydroflumethiazide is administered in an amount of about 25 milligrams to about 200 milligrams as a single dose or as multiple doses per day; the triamterene is administered in an amount of about 35 milligrams to about 225 milligrams as a single dose or as multiple doses per day.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient a therapeutically effective amount of (i) at least one hydralazine compound or a pharmaceutically acceptable salt thereof (e.g., preferably hydralazine hydrochloride), (ii) at least one of isosorbide dinitrate and isosorbide mononitrate (e.g., preferably isosorbide dinitrate), and (iii) a cardiac glycoside. The compounds can be administered separately or in the form of a composition. In one embodiment the cardiac glycoside is digoxin, acetyldigoxin, deslanoside, digitoxin or medigoxin. In other embodiments the digoxin is administered to achieve a steady state blood serum concentration of at least about 0.7 nanograms per ml to about 2.0 nanograms per ml.

The invention provides methods for (a) prolonging time to hospitalization for heart failure; (b) prolonging time to first hospitalization for heart failure; (c) reducing the total number of days a patient with heart failure spends in the hospital for heart failure for a single hospital stay (i.e., reducing the duration of a single hospital stay for a patient with heart failure); (d) reducing the total number of days a patient spends in the hospital for heart failure for multiple hospital stays (i.e., two or more hospital stays); (e) reducing the number of hospital admissions for heart failure; (f) reducing mortality and reducing hospitalizations for heart failure (e.g., the total number of days in the hospital and/or the number of hospital visits); (g) increasing the left ventricular ejection fraction in a heart failure patient; (h) treating a sexual dysfunction (e.g., erectile dysfunction and female sexual dysfunction) (j) treating a headache in a heart failure patient by administering a non-steroidal antiinflammatory compound (i.e., NSAIDs); (k) treating a heart failure patient who has a history of hypertension (but who is not currently diagnosed with hypertension); (l) improving the quality of life in a heart failure patient based on the Minnesota Living with heart failure questionnaire; (m) decreasing the levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; and (u) reducing cardiomegaly in a patient in need thereof comprising administering to the patient a therapeutically effective amount of (i) a hydralazine compound (preferably hydralazine hydrochloride), (ii) isosorbide dinitrate and/or isosorbide mononitrate (preferably isosorbide dinitrate), (iii) an angiotensin-converting enzyme inhibitor selected from the group consisting of captopril, enalapril, lisinopril, trandolapril and trandolaprilat and (iv) a β-adrenergic antagonist selected from the group consisting of carvedilol, metoprolol, bisoprolol and nebivolol. In another embodiment, the invention provides methods of administering (i) a hydralazine compound (preferably hydralazine hydrochloride), (ii) isosorbide dinitrate and/or isosorbide mononitrate (preferably isosorbide dinitrate), (iii) an angiotensin-converting enzyme inhibitor selected from the group consisting of enalapril, lisinopril, trandolapril and trandolaprilat and (iv) an aldosterone antagonist selected from the group consisting of eplerenone and spironolactone. In another embodiment, the invention provides methods of administering (i) a hydralazine compound (preferably hydralazine hydrochloride), (ii) isosorbide dinitrate and/or isosorbide mononitrate (preferably isosorbide dinitrate), (iii) an angiotensin-converting enzyme inhibitor selected from the group consisting of captopril, enalapril, lisinopril, trandolapril and trandolaprilat and (iv) an angiotensin II antagonist selected from the group consisting of losartan, candesartan, irbesartan and valsartan. In another embodiment, the invention provides methods of administering (i) a hydralazine compound (preferably hydralazine hydrochloride), (ii) isosorbide dinitrate and/or isosorbide mononitrate (preferably isosorbide dinitrate), (iii) a β-adrenergic antagonist selected from the group consisting of carvedilol, metoprolol, bisoprolol and nebivolol and (iv) an aldosterone antagonist selected from the group consisting of eplerenone and spironolactone. In another embodiment, the invention provides methods of administering (i) a hydralazine compound (preferably hydralazine hydrochloride), (ii) isosorbide dinitrate and/or isosorbide mononitrate (preferably isosorbide dinitrate), (iii) a β-adrenergic antagonist selected from the group consisting of carvedilol, metoprolol, bisoprolol and nebivolol and (iv) an angiotensin II antagonist selected from the group consisting of losartan, candesartan, irbesartan and valsartan. In another embodiment, the invention provides methods of administering (i) a hydralazine compound (preferably hydralazine hydrochloride), (ii) isosorbide dinitrate and/or isosorbide mononitrate (preferably isosorbide dinitrate), (iii) an angiotensin II antagonist selected from the group consisting of losartan, candesartan, irbesartan and valsartan (iv) a β-adrenergic antagonist selected from the group consisting of carvedilol, metoprolol, bisoprolol and nebivolol and (v) an aldosterone antagonist selected from the group consisting of eplerenone and spironolactone. In another embodiment, the invention provides methods of administering (i) a hydralazine compound (preferably hydralazine hydrochloride), (ii) isosorbide dinitrate and/or isosorbide mononitrate (preferably isosorbide dinitrate), (iii) an angiotensin-converting enzyme inhibitor selected from the group consisting of captopril, enalapril, lisinopril, trandolapril and trandolaprilat (iv) a β-adrenergic antagonist selected from the group consisting of carvedilol, metoprolol, bisoprolol and nebivolol and (v) an angiotensin II antagonist selected from the group consisting of losartan, candesartan, irbesartan and valsartan. In another embodiment, the invention provides methods of administering (i) a hydralazine compound (preferably hydralazine hydrochloride), (ii) isosorbide dinitrate and/or isosorbide mononitrate (preferably isosorbide dinitrate), (iii) an angiotensin II antagonist selected from the group consisting of losartan, candesartan, irbesartan and valsartan and (iv) an aldosterone antagonist selected from the group consisting of eplerenone and spironolactone. In another embodiment, the patient is black. In another embodiment, the patient with heart failure is categorized as New York Heart Association (NYHA) heart failure functional classification I, II, III or IV. In these embodiments the hydralazine compound, and at least one of isosorbide dinitrate and isosorbide mononitrate can be administered separately or as components of the same composition, and can be administered in the form of a composition with or simultaneously with, subsequently to, or prior to administration of at least one of the angiotensin converting enzyme inhibitor, β-adrenergic antagonist, angiotensin II antagonist, aldosterone antagonist, or combinations of two or more thereof. In one embodiment, all the compounds are administered together in the form of a single composition.

In one embodiment, the invention provides methods for treating a headache in a heart failure patient in need thereof comprising administering to the patient a therapeutically effective amount of (i) at least one hydralazine compound or a pharmaceutically acceptable salt thereof (e.g., preferably hydralazine hydrochloride), (ii) at least one of isosorbide dinitrate and isosorbide mononitrate (e.g., preferably isosorbide dinitrate), and (iii) a non-steroidal anti-inflammatory compound. Suitable NSAIDs include, but are not limited to, acetaminophen, acemetacin, aceclofenac, alminoprofen, amfenac, bendazac, benoxaprofen, bromfenac, bucloxic acid, butibufen, carprofen, cinmetacin, clopirac, diclofenac, etodolac, felbinac, fenclozic acid, fenbufen, fenoprofen, fentiazac, flunoxaprofen, flurbiprofen, ibufenac, ibuprofen, indomethacin, isofezolac, isoxepac, indoprofen, ketoprofen, lonazolac, loxoprofen, metiazinic acid, mofezolac, miroprofen, naproxen, oxaprozin, pirozolac, pirprofen, pranoprofen, protizinic acid, salicylamide, sulindac, suprofen, suxibuzone, tiaprofenic acid, tolmetin, xenbucin, ximoprofen, zaltoprofen, zomepirac, aspirin, acemetcin, bumadizon, carprofenac, clidanac, diflunisal, enfenamic acid, fendosal, flufenamic acid, flunixin, gentisic acid, ketorolac, meclofenamic acid, mefenamic acid, mesalamine, prodrugs thereof, and the like. Suitable NSAIDs are described more fully in the literature, such as in Goodman and Gilman, The Pharmacological Basis of Therapeutics (9th Edition), McGraw-Hill, 1995, Pgs. 617-657; the Merck Index on CD-ROM, 13^{th} Edition; and in U.S. Pat. Nos. 6,057,347 and 6,297,260 assigned to NitroMed. Inc., the disclosures of which are incorporated herein by reference in their entirety.

In some embodiments the NSAIDs are acetaminophen, diclofenac, flurbiprofen, ibuprofen, indomethacin, ketoprofen, naproxen or aspirin. In other embodiments the acetaminophen is administered in an amount of about 325 milligrams to about 6 grams as a single dose or as multiple doses per day; the diclofenac is administered in an amount of about 50 milligrams to about 250 milligrams as a single dose or as multiple doses per day; the flurbiprofen is administered in an amount of about 100 milligrams to about 300 milligrams as a single dose or as multiple doses per day; the ibuprofen is administered in an amount of about 400 milligrams to about 3.2 grams as a single dose or as multiple doses per day; the indomethacin is administered in an amount of about 25 milligrams to about 200 milligrams as a single dose or as multiple doses per day; the ketoprofen is administered in an amount of about 50 milligrams to about 300 milligrams as a single dose or as multiple doses per day; the naproxen is administered in an amount of about 250 milligrams to about 1.5 grams as a single dose or as multiple doses per day; the aspirin is administered in an amount of about 10 milligrams to about 2 grams as a single dose or as multiple doses per day.

In one embodiment, the invention provides methods for treating a headache in a heart failure patient in need thereof comprising administering to the patient hydralazine hydrochloride in an amount of about 30 milligrams to about 300 milligrams per day, isosorbide dinitrate in an amount of about 20 milligrams to about 200 milligrams per day and acetaminophen in an amount of about 375 milligrams to about 6000 milligrams per day. In another embodiment the patient is administered hydralazine hydrochloride in an amount of about 75 milligrams to about 225 milligrams per day, isosorbide dinitrate in an amount of about 40 milligrams to about 120 milligrams per day and acetaminophen in an amount of about 500 milligrams to about 4000 milligrams per day. In this embodiment the hydralazine may be administered as 75 mg once, twice or three times per day, the isosorbide dinitrate may be administered as 40 mg once, twice or three times per day, and the acetaminophen may be administered as 500 milligrams to about 1000 milligrams one to four times per day. In another embodiment the patient is administered hydralazine hydrochloride in an amount of about 37.5 milligrams to about 112.5 milligrams per day, isosorbide dinitrate in an amount of about 20 milligrams to about 60 milligrams per day and acetaminophen in an amount of about 500 milligrams to about 4000 milligrams per day. In this embodiment the hydralazine may be administered as 37.5 mg once, twice or three times per day, the isosorbide dinitrate may be administered as 20 mg once, twice or three times per day and the acetaminophen may be administered as 500 milligrams to about 1000 milligrams once, twice, three or four times per day. In these embodiments the hydralazine hydrochloride, isosorbide dinitrate and acetaminophen, can be administered separately or in the form of a composition.

The compounds and compositions of the invention can be administered by any available and effective delivery system including, but not limited to, orally, bucally, parenterally, by inhalation spray, or topically (including transdermally), in dosage unit formulations containing conventional nontoxic pharmaceutically acceptable carriers, adjuvants, and vehicles as desired. The preferred methods of administration of the compounds and compositions are by oral administration.

When administered in vivo, the compounds and compositions of the invention, can be administered in combination with pharmaceutically acceptable carriers and in dosages described herein. The compounds and compositions of the invention can also be administered in combination with one or more additional compounds which are known to be effective for the treatment of heart failure or other diseases or disorders, such as, for example, anti-hyperlipidemic compounds, such as, for example, statins or HMG-CoA reductase inhibitors, such as, for example, atorvastatin (LIPITOR®), bervastatin, cerivastatin (BAYCOL®), dalvastatin, fluindostatin (Sandoz XU-62-320), fluvastatin, glenvastatin, lovastatin (MEVACOR®), mevastatin, pravastatin (PRAVACHOL®), rosuvastatin (CRESTRO®), simvastatin (ZOCOR®), velostatin (also known as synvinolin), VYTORIN™ (ezetimibe/simvastatin), GR-95030, SQ 33,600, BMY 22089, BMY 22,566, CI 980, and the like; gemfibrozil, cholystyramine, colestipol, niacin, nicotinic acid, bile acid sequestrants, such as, for example, cholestyramine, colesevelam, colestipol, poly(methyl-(3-trimethylaminopropyl) imino-trimethylene dihalide) and the like; probucol; fibric acid agents or fibrates, such as, for example, bezafibrate (Bezalip™), beclobrate, binifibrate, ciprofibrate, clinofibrate, clofibrate, etofibrate, fenofibrate (Lipidil™, Lipidil Micro™), gemfibrozil (Lopid™), nicofibrate, pirifibrate, ronifibrate, simfibrate, theofibrate and the like; cholesterol ester transfer protein (CETP) inhibitors, such as for example, CGS 25159, CP-529414 (torcetrapid), JTT-705, substituted N-[3-(1,1,2,2-tetrafluoroethoxy)benzyl]-N-(3-phenoxyphenyl)-trifluoro-3-amino-2-propanols, N,N-disubstituted trifluoro-3-amino-2-propanols, PD 140195 (4-phenyl-5-tridecyl-4H-1,2,4-triazole-3-thiol), SC-794, SC-795, SCH 58149, and the like. The hydralazine compound or pharmaceutically acceptable salt thereof, and the at least one of isosorbide dinitrate and isosorbide mononitrate, can be administered simultaneously with, subsequently to, or prior to administration of the anti-hyperlipidemic compound, or they can be administered in the form of a composition.

Solid dosage forms for oral administration can include capsules, tablets, effervescent tablets, chewable tablets, pills, powders, sachets, granules and gels. In such solid dosage forms, the active compounds can be admixed with at least one inert diluent such as, sucrose, lactose or starch. Such dosage forms can also comprise, as in normal practice, additional substances other than inert diluents, e.g., lubricating agents such as, magnesium stearate. In the case of capsules, tablets, effervescent tablets, and pills, the dosage forms can also comprise buffering agents. Soft gelatin capsules can be prepared to contain a mixture of the active compounds or compositions of the invention and vegetable oil. Hard gelatin capsules can contain granules of the active compound in combination with a solid, pulverulent carrier such as, lactose, saccharose, sorbitol, mannitol, potato starch, corn starch, amylopectin, cellulose derivatives of gelatin. Tablets and pills can be prepared with enteric coatings. Oral formulations containing compounds of the invention are disclosed in U.S. Pat. Nos. 5,559,121, 5,536,729, 5,989,591 and 5,985,325, the disclosures of each of which are incorporated by reference herein in their entirety.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

Suppositories for vaginal or rectal administration of the compounds and compositions of the invention can be prepared by mixing the compounds or compositions with a suitable nonirritating excipient such as, cocoa butter and polyethylene glycols which are solid at room temperature but liquid at body temperature, such that they will melt and release the drug.

Injectable preparations, for example, sterile injectable aqueous or oleaginous suspensions can be formulated according to the known art using suitable dispersing agents, wetting agents and/or suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. Sterile fixed oils are also conventionally used as a solvent or suspending medium. Parenteral formulations containing compounds of the invention are disclosed in U.S. Pat. Nos. 5,530,006, 5,516,770 and 5,626,588, the disclosures of each of which are incorporated by reference herein in their entirety.

Transdermal compound administration, which is known to one skilled in the art, involves the delivery of pharmaceutical compounds via percutaneous passage of the compound into the systemic circulation of the patient. Topical administration can also involve the use of transdermal administration such as, transdermal patches or iontophoresis devices. Other components can be incorporated into the transdermal patches as well. For example, compositions and/or transdermal patches can be formulated with one or more preservatives or bacteriostatic agents including, but not limited to, methyl hydroxybenzoate, propyl hydroxybenzoate, chlorocresol, benzalkonium chloride, and the like. Dosage forms for topical administration of the compounds and compositions can include creams, pastes, sprays, lotions, gels, ointments, and the like. In such dosage forms, the compositions of the invention can be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the compositions can contain polyethylene glycol 400. They can be mixed to form ointments with, for example, benzyl alcohol 2% (wt/wt) as preservative, white petrolatum, emulsifying wax, and tenox II (butylated hydroxyanisole, propyl gallate, citric acid, propylene glycol). Woven pads or rolls of bandaging material, e.g., gauze, can be impregnated with the compositions in solution, lotion, cream, ointment or other such form can also be used for topical application. The compositions can also be applied topically using a transdermal system, such as one of an acrylic-based polymer adhesive with a resinous crosslinking agent impregnated with the composition and laminated to an impermeable backing.

The compositions of this invention can further include conventional excipients, i.e., pharmaceutically acceptable organic or inorganic carrier substances suitable for parenteral application which do not deleteriously react with the active compounds. Suitable pharmaceutically acceptable carriers include, for example, water, salt solutions, alcohol, vegetable oils, polyethylene glycols, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like. The pharmaceutical preparations can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds. For parenteral application, particularly suitable vehicles consist of solutions, preferably oily or aqueous solutions, as well as suspensions, emulsions, or implants. Aqueous suspensions may contain substances that increase the viscosity of the suspension and include, for example, sodium carboxymethyl cellulose, sorbitol and/or dextran. Optionally, the suspension may also contain stabilizers.

Solvents useful in the practice of this invention include pharmaceutically acceptable, water-miscible, non-aqueous solvents. In the context of this invention, these solvents should be taken to include solvents that are generally acceptable for pharmaceutical use, substantially water-miscible, and substantially non-aqueous. The pharmaceutically-acceptable, water-miscible, non-aqueous solvents usable in the practice of this invention include, but are not limited to, N-methylpyrrolidone (NMP); propylene glycol; ethyl acetate; dimethyl sulfoxide; dimethyl acetamide; benzyl alcohol; 2-pyrrolidone; benzyl benzoate; C₂₋₆ alkanols; 2-ethoxyethanol; alkyl esters such as, 2-ethoxyethyl acetate, methyl acetate, ethyl acetate, ethylene glycol diethyl ether, or ethylene glycol dimethyl ether; (S)-(−)-ethyl lactate; acetone; glycerol; alkyl ketones such as, methylethyl ketone or dimethyl sulfone; tetrahydrofuran; cyclic alkyl amides such as, caprolactam; decylmethylsulfoxide; oleic acid; aromatic amines such as, N,N-diethyl-m-toluamide; or 1-dodecylazacycloheptan-2-one.

The preferred pharmaceutically-acceptable, water-miscible, non-aqueous solvents are N-methylpyrrolidone (NMP), propylene glycol, ethyl acetate, dimethyl sulfoxide, dimethyl acetamide, benzyl alcohol, 2-pyrrolidone, or benzyl benzoate. Ethanol may also be used as a pharmaceutically-acceptable, water-miscible, non-aqueous solvent according to the invention, despite its negative impact on stability. Additionally, triacetin may also be used as a pharmaceutically-acceptable, water-miscible, non-aqueous solvent, as well as functioning as a solubilizer in certain circumstances. NMP may be available as PHARMASOLVE® from International Specialty Products (Wayne, N.J.). Benzyl alcohol may be available from J. T. Baker, Inc. Ethanol may be available from Spectrum, Inc. Triacetin may be available from Mallinckrodt, Inc.

The compositions of this invention can further include solubilizers. Solubilization is a phenomenon that enables the formation of a solution. It is related to the presence of amphiphiles, that is, those molecules that have the dual properties of being both polar and non-polar in the solution that have the ability to increase the solubility of materials that are normally insoluble or only slightly soluble, in the dispersion medium. Solubilizers often have surfactant properties. Their function may be to enhance the solubility of a solute in a solution, rather than acting as a solvent, although in exceptional circumstances, a single compound may have both solubilizing and solvent characteristics. Solubilizers useful in the practice of this invention include, but are not limited to, triacetin, polyethylene glycols (such as, for example, PEG 300, PEG 400, or their blend with 3350, and the like), polysorbates (such as, for example, Polysorbate 20, Polysorbate 40, Polysorbate 60, Polysorbate 65, Polysorbate 80, and the like), poloxamers (such as, for example, Poloxamer 124, Poloxamer 188, Poloxamer 237, Poloxamer 338, Poloxamer 407, and the like), polyoxyethylene ethers (such as, for example, Polyoxyl 2 cetyl ether, Polyoxyl 10 cetyl ether, and Polyoxyl 20 cetyl ether, Polyoxyl 4 lauryl ether, Polyoxyl 23 lauryl ether, Polyoxyl 2 oleyl ether, Polyoxyl 10 oleyl ether, Polyoxyl 20 oleyl ether, Polyoxyl 2 stearyl ether, Polyoxyl 10 stearyl ether, Polyoxyl 20 stearyl ether, Polyoxyl 100 stearyl ether, and the like), polyoxylstearates (such as, for example, Polyoxyl 30 stearate, Polyoxyl 40 stearate, Polyoxyl 50 stearate, Polyoxyl 100 stearate, and the like), polyethoxylated stearates (such as, for example, polyethoxylated 12-hydroxy stearate, and the like), and Tributyrin.

Other materials that may be added to the compositions of the invention include cyclodextrins, and cyclodextrin analogs and derivatives, and other soluble excipients that could enhance the stability of the inventive composition, maintain the product in solution, or prevent side effects associated with the administration of the inventive composition. Cyclodextrins may be available as ENCAPSIN® from Janssen Pharmaceuticals.

The composition, if desired, can also contain minor amounts of wetting agents, emulsifying agents and/or pH buffering agents. The composition can be a liquid solution, suspension, emulsion, tablet, pill, capsule, sustained release formulation, or powder. The composition can be formulated as a suppository, with traditional binders and carriers such as, triglycerides. Oral formulations can include standard carriers such as, pharmaceutical grades of mannitol, lactose, starch, magnesium stearate, sodium saccharine, cellulose, magnesium carbonate, and the like.

Various delivery systems are known and can be used to administer the compounds or compositions of the invention, including, for example, encapsulation in liposomes, microbubbles, emulsions, microparticles, microcapsules, nanoparticles, and the like. The required dosage can be administered as a single unit or in a sustained release form.

The bioavailability of the compositions can be enhanced by micronization of the formulations using conventional techniques such as, grinding, milling, spray drying and the like in the presence of suitable excipients or agents such as, phospholipids or surfactants.

The compounds and compositions of the invention can be formulated as pharmaceutically acceptable salts. Pharmaceutically acceptable salts include, for example, alkali metal salts and addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids include, but are not limited to, hydrochloric, hydrobromic, hydroiodic, nitrous (nitrite salt), nitric (nitrate salt), carbonic, sulfuric, phosphoric acid, and the like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, β-hydroxybutyric, cyclohexylaminosulfonic, galactaric and galacturonic acid and the like. Suitable pharmaceutically-acceptable base addition salts include, but are not limited to, metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, meglumine (N-methylglucamine) and procaine and the like. All of these salts may be prepared by conventional means from the corresponding compound by reacting, for example, the appropriate acid or base with the compound.

While individual needs may vary, determination of optimal ranges for effective amounts of the compounds and/or compositions is within the skill of the art and can be determined by standard clinical techniques, including reference to Goodman and Gilman, supra; The Physician's Desk Reference, Medical Economics Company, Inc., Oradell, N.J., 1995; and Drug Facts and Comparisons, Inc., St. Louis, Mo., 1993. Generally, the dosage required to provide an effective amount of the compounds and compositions, which can be adjusted by one of ordinary skill in the art, will vary depending on the age, health, physical condition, sex, diet, weight, extent of the dysfunction of the recipient, frequency of treatment and the nature and scope of the dysfunction or disease, medical condition of the patient, the route of administration, pharmacological considerations such as, the activity, efficacy, pharmacokinetic and toxicology profiles of the particular compound used, whether a drug delivery system is used, and whether the compound is administered as part of a drug combination.

### EXAMPLES

The following examples are for purposes of illustration only and are not intended to limit the spirit or scope of the appended claims.

### Example 1

### Summary of Protocol for the African-American Heart Failure Trial (A-HeFT)

### Study Design:

1. Open study to African-Americans (AFA) with moderate to severe, stable symptomatic heart failure (HF) (NYHA class III-IV), and left ventricular dysfunction [Left ventricle ejection fraction, LVEF≦35%, or left ventricle diastolic internal dimension, LVIDD>2.9 cm/m², body surface area BSA (or >6.5 cm) with LVEF<45%] while on standard therapy (e.g., ACE-I, digitalis, diuretic and/or beta blocker).

2. Randomization—parallel groups, double blind, stratified for beta blocker usage.

3. Study drugs—37.5 mg hydralazine hydrochloride and 20 mg isosorbide dinitrate per tablet or placebo tablets t.i.d., with forced titration to maximum dose of 225 mg/day of hydralazine hydrochloride and 120 mg/day of isosorbide dinitrate (maximum dose=2 tablets t.i.d.).

4. Study duration—Randomization rate driven, i.e., all patients treated and followed for either a maximum of 18 months or until the last patient randomized has completed 6 months post-randomization, whichever occurs first.

5. Scheduled visits—screening, baseline following 2-4 weeks of stabilization (randomize at baseline if eligible), every 3 months thereafter for either a maximum of 18 months or until the last patient randomized has completed 6 months post-randomization, whichever occurs first.

6. Observations/procedures—history & physical, New York Heart Association (NYHA) class, echocardiogram (for LVEF and LVIDD, read blinded by a central laboratory), quality of life (QOL) assessment, safety lab profile (routine at baseline only, PRN thereafter).

### Objectives:

The overall objective was to demonstrate safety and efficacy of the combination of hydralazine hydrochloride and isosorbide dinitrate versus placebo in patients with moderate to severe symptomatic HF (NYHA class III-IV) receiving standard treatment. The specific objectives were:

1. To demonstrate statistically significant superiority of the combination of hydralazine hydrochloride and isosorbide dinitrate compared to placebo in the primary efficacy endpoint consisting of a composite score calculated from change in QOL measurement, and clinical outcomes including hospitalizations for heart failure and deaths.

2. To confirm the safety and tolerability of the combination of hydralazine hydrochloride and isosorbide dinitrate in patients with HF.

3. To achieve favorable trends in one or more secondary endpoints consisting of the individual components of the primary endpoint composite, total number of hospitalizations, duration of hospitalizations, unscheduled office and emergency room visits, and measures of cardiac size and function.

### Study Population:

1. The criteria for inclusion in the study included: (a) African-American (self identified)>18 years of age, either sex; (b) in-patient or out-patient; stable, symptomatic HF, NYHA class III or IV; (c) Background treatment—Standard treatment, including ACE-I, digitalis, diuretics, beta blockers, angiotensin II antagonists, aldosterone antagonists and/or spironolactone as needed. Patients taking beta blockers must have been receiving them for at least 3 months before enrollment; (d) LVEF≦35%, or LVIDD>2.9 cm/m² BSA (or >6.5 cm) with LVEF<45% by echocardiogram anytime within the prior 6 months, using the most recent echo available; and (e) HF symptoms and treatment regimen stable for at least 2 weeks (diuretics may be adjusted during this interval, but other HF medications should not be changed).

2. The criteria for exclusion from the study included: (a) significant valvular heart disease; (b) cardiac transplantation likely to be required within 1 year; (c) uncontrolled hypertension; (d) significant hepatic, renal, or other disease which might limit survival or exercise capacity; (e) history of cardiac arrest within 3 months unless treated with implantable cardioverter defibrillator (ICD); (f) received parenteral inotropic therapy within past 1 month; (g) the need for the following medications—hydralazine, long-acting nitrates, or VIAGRA® (Pfizer, Inc.), LEVITRA® (Bayer Pharmaceuticals Corporation) or CIALIS® (Lilly ICOS Inc.).

### Endpoints:

1. Primary endpoints

TABLE 1
Scoring System for the Primary Composite End Points

| End Point | Score |
|---|---|
| Death (at any time during the trial) | −3 |
| Survival to end of trial | 0 |
| First hospitalization for heart failure (adjudicated) | −1 |
| No hospitalization | 0 |
| Change in quality of life at 6 months | |
| (or at last measurement if earlier than 6 months) | |
| Improvement in quality of life by ≧10 units | +2 |
| Improvement in quality of life by 5-9 units | +1 |
| Change in quality of life by <5 units | 0 |
| Worsening in quality of life by 5-9 units | −1 |
| Worsening in quality of life by ≦10 units | −2 |
| Possible Score | −6 to +2 |

1. Secondary endpoints:
   a. Individual components of the primary endpoint composite and LVEF
      i. Deaths (all causes; cardiac)
      ii. Number of hospitalizations (all causes; heart failure related)
      iii. QOL
      iv. LVEF
   b. Other
      i. Days in hospital
      ii. ER visits and unscheduled office visits
      iii. LVIDD and LV wall thickness
      iv. Newly recognized need for cardiac transplantation (Patients actually undergoing cardiac transplantation during the trial will have their data censored at the time of transplantation.)
      v. Change in B-type natriuretic peptide (BNP) at six months.

### Schedule of Visits and Observations:

1. Visit −1=Screening
   a. History & physical for screening, inclusion/exclusion criteria
   b. Adjust background therapy as needed
   c. Baseline safety labs
   d. Confirm LVEF≦35%, or LVIDD>2.9 cm/m² BSA (or >6.5 cm) with LVEF<45% by latest available echocardiogram within past 6 months
   e. Schedule return visit in 2-4 weeks
2. Visit 0=Baseline
   a. Confirm patient eligibility
      1. Stable for past 2-4 weeks (symptoms, medications, and weight)
      2. Lab tests
   b. Baseline assessments
      1. QOL
      2. Echocardiogram for LVEF and LVIDD. (Note: This echocardiogram is used for baseline values of LVEF and LVIDD. The patient remains in the study even if these baseline LVEF and/or LVIDD values no longer meet inclusion criteria, provided all other eligibility criteria are still satisfied.)
      3. NYHA class
      4. History of morbid events during prior 6 months
   c. Concomitant medications
   d. Randomize patient, double-blind, to a combination of hydralazine hydrochloride and isosorbide dinitrate or placebo.
   e. Start study drug, 1 tablet t.i.d., within 24 hours of Visit 0.
3. Dose titration follow-up=3-5 days after randomization
   a. Telephone call or clinic visit (per investigator discretion)
   b. If study medication is well tolerated, patient is force titrated to 2 tablets t.i.d.
   c. If study medication is not well tolerated the investigator may make appropriate dosage adjustments of study medication and/or background medications as clinically indicated
   d. Schedule further dose-titration follow-up in 3-5 days as needed
4. Visit 1=month 3 after randomization
   a. QOL
   b. Interim history and brief physical
   c. NYHA class
   d. History of morbid events since last visit
   e. Concomitant medications
   f. Adverse events
5. Visit 2=month 6 after randomization
   a. QOL
   b. History and physical
   c. LVEF and LVIDD (echocardiogram)
   d. NYHA class
   e. History of morbid events since last visit
   f. Concomitant medications
   g. Adverse events
6. Visits 3+=month 9 after randomization and every 3 months thereafter for either a maximum of 18 months or until the last patient randomized has completed 6 months post-randomization, whichever occurs first.
   a. QOL [NOTE: Data to be used only for secondary endpoint analyses. For primary composite endpoint only QOL data at 6 months (or sooner, if 6 month QOL not available) will be used.]
   b. Interim history and brief physical
   c. NYHA class
   d. History of morbid events since last visit
   e. Concomitant medications
   f. Adverse events
7. Last Visit on Study Drug. Performed in any patient who is terminated from the study anytime before Visit 2 (6 months), and all remaining patients at the time of overall study termination who have not had any assessments within the 2 weeks preceding study termination.
   a. QOL
   b. History and physical
   c. NYHA class
   d. History of morbid events since last visit
   e. Concomitant medications
   f. Adverse events

### Baseline Characteristics

A total of 1050 patients were randomized, 532 to placebo and 518 to a combination of hydralazine hydrochloride and isosorbide dinitrate.

The patients enrolled were middle-aged men and women (Table 2). The most common cause of heart failure was hypertensive heart disease; less than one-fourth of the patients had heart failure due to ischemic heart disease. More than 90% of the patients had NYHA class III symptoms. In general, the two treatment groups were well-matched for baseline characteristics; more men were randomized to the placebo group (p=0.01) and baseline diastolic blood pressure was higher in the hydralazine hydrochloride/isosorbide dinitrate group (p=0.002).

The baseline demographic and clinical characteristics are summarized in Table 2.

TABLE 2

| | Hydralazine | | | |
|---|---|---|---|---|
| | hydrochloride and | | | |
| | Isosorbide Dinitrate | | Placebo | |
| | (N = 518) | | (N = 532) | |
| Age (years) | 56.8 | (12.7) | 56.9 | (13.3) |
| Sex, men/women (n) | 290/228† | | 340/192 | |
| Etiology of heart failure, n (%) | | | | |
| Ischemic | 121 | (23.4) | 121 | (22.7) |
| Idiopathic | 127 | (24.5) | 147 | (27.6) |
| Hypertensive | 207 | (40.0) | 199 | (37.4) |
| Valvular | 13 | (2.5) | 17 | (3.2) |
| Other | 50 | (9.7) | 48 | (9.0) |
| Ejection fraction, %, mean (SD) | 23.9 | (7.3) | 24.2 | (7.5) |
| | n = 517 | | n = 532 | |
| Left ventricular internal diastolic | 6.5 | (0.9) | 6.5 | (1.0) |
| dimension (cm), Mean SD | n = 330 | | n = 332 | |
| Baseline NYHA class, n (%) | | | | |
| I | 1 | (0.2) | 1 | (0.2) |
| II | 9 | (1.7) | 2 | (0.4) |
| III | 493 | (95.2) | 503 | (94.7) |
| IV | 15 | (2.9) | 25 | (4.7) |
| Missing | 0 | (0.0) | 1 | (0.2) |
| Systolic blood pressure, | 127.2 | (17.5) | 125.3 | (18.1) |
| mmHg mean (SD) | | | | |
| Diastolic blood pressure, | 77.6 | (10.3)† | 75.6 | (10.6) |
| mmHg mean (SD) | | | | |
| Heart rate, beats/min Mean (SD) | 74.2 | (12.3) | 73.1 | (11.0) |

†p < 0.05 relative to placebo

Approximately 90% of the patients enrolled had a history of hypertension, 53% had hyperlipidemia, and 41% had diabetes mellitus (Table 3). With respect to cardiovascular history, the groups were well-matched except for hyperlipidemia and diabetes mellitus, which were more frequent in the hydralazine hydrochloride and isosorbide dinitrate-treated patients (p=0.04 and 0.012, respectively).

The majority of the patients were taking diuretics (92%), beta blockers (83%), angiotensin-converting enzyme inhibitors (75%), anti-thrombotic agents (72%) and digitalis glycosides (60%). The two groups were similar with respect to baseline medications, except for the more frequent use of anti-diabetic medications in the hydralazine hydrochloride and isosorbide dinitrate group.

The baseline cardiovascular history and treatment are summarized in Table 3.

TABLE 3

| | Hydralazine | |
|---|---|---|
| | hydrochloride and | |
| | Isosorbide | |
| | Dinitrate | Placebo |
| | (N = 518) | (N = 532) |
| Cardiovascular history (n, %) | | |
| History of hypertension | 472 (91.1) | 468 (88.0) |
| Arrhythmias | 169 (32.6) | 184 (34.6) |
| Diabetes mellitus | 232 (44.8) | 197 (37.0) |
| Hyperlipidemia | 289 (55.8) | 263 (49.4) |
| Cerebrovascular disease | 79 (15.3) | 74 (13.9) |
| Peripheral vascular disease | 58 (11.2) | 71 (13.3) |
| Chronic obstructive lung disease | 91 (17.6) | 110 (20.7) |
| Chronic renal insufficiency | 84 (16.2) | 97 (18.2) |
| Valvular disease | 186 (35.9) | 194 (36.5) |
| Previous revascularization | 111 (21.4) | 96 (18.0) |
| Pacemaker or implantable defibrillator | 86 (16.6) | 92 (17.3) |
| Previous myocardial infarction | 152 (29.3) | 152 (28.6) |
| Current angina | 75 (14.5) | 78 (14.7) |
| Current smoking | 143 (27.6) | 140 (26.3) |
| Previous smoking | 306 (59.1) | 336 (63.2) |
| Background medications (n, %) | | |
| Diuretics | 473 (91.3) | 494 (92.9) |
| Angiotensin-converting enzyme inhibitors | 386 (74.5) | 400 (75.2) |
| Angiotensin receptor blockers | 124 (23.9) | 112 (21.1) |
| Beta blockers | 434 (83.8) | 437 (82.1) |
| Calcium channel blockers | 109 (21.0) | 104 (19.5) |
| Digitalis glycosides | 304 (58.7) | 324 (60.9) |
| Aldosterone antagonists | 208 (40.2) | 201 (37.8) |
| Anti-arrhythmics class I and III | 52 (10.0) | 62 (11.7) |
| Anti-thrombotic agents | 380 (73.4) | 381 (71.6) |
| Lipid lowering agents | 219 (42.3) | 206 (38.7) |
| Insulin | 97 (18.7) | 67 (12.6) |
| Oral hypoglycemic drugs | 156 (30.1) | 119 (22.4) |
| Potassium supplement | 256 (49.4) | 271 (50.9) |

### Example 2

### Results

Analysis of the results after the enrollment of 1050 of the 1100 patients demonstrated a statistically significant favorable mortality benefit for patients administered a combination of hydralazine hydrochloride and isosorbide dinitrate (treatment group) when compared to those that were administered placebo (control group).

Additional descriptive statistics were estimated for patient characteristics and reported as means (±SDs) or counts (and percentages). Adverse events were also compared between groups using chi-square tests.

The primary efficacy comparison included all participants who had been randomized at the time of the termination of the trial. For missing data, the worst case score (i.e., −6) for that component was assumed for the calculation of the primary analysis. The composite end point was compared between groups with the use of a two-sample t test.

There were 54 deaths (10.2%) in the control group and 32 deaths in the treatment group (6.2%) that showed a 43% reduction in mortality for the treatment group. p=0.001 when adjusted for repeated looks (interim analyses). This mortality benefit has trended consistently in favor of treatment over the last 3 Data and Safety Monitoring Board (DSMB) meetings.

Data available on change in quality of life and hospitalization for heart failure are consistent with these mortality results: Mean change in quality of life was −2.7 for the control group and −5.6 for the treatment group; p=0.02. This indicated more improvement for the treatment group. 130 patients (24.4%) in the control group were hospitalized for heart failure as opposed to 85 patients (16.4%) in the treatment group, for a 39% decrease. p=0.001.

### Patient Disposition and Exposure to Study Medication

The duration of a patient's participation in the trial was longer for those treated with hydralazine hydrochloride and isosorbide dinitrate (379 days) than for those treated with placebo (355 days), p=0.04. This difference was due to the higher withdrawal rate from the study for placebo patients than for the hydralazine hydrochloride and isosorbide dinitrate patients (14.1% vs 9.5%), largely due to a higher withdrawal rate for death in placebo patients (10.2% vs 6.2%).

In contrast, the duration of exposure to the study drug was shorter in the hydralazine hydrochloride and isosorbide dinitrate-treated patients than in placebo-treated patients (298 days vs 314 days). This difference was related to the higher frequency of withdrawals for adverse events in the hydralazine hydrochloride and isosorbide dinitrate-treated patients than placebo-treated patients (21.1% vs 12.0%).

As shown in Table 4, patients were more likely to remain on treatment with placebo than on treatment with hydralazine hydrochloride and isosorbide dinitrate at each time point in the trial.

TABLE 4
Patients on Study Drug at Various Time Points [n (%)]

| | hydralazine hydrochloride and | |
|---|---|---|
| | isosorbide dinitrate | |
| Time on Study | (n = 517) | Placebo (n = 527) |
| 3 months | 368 (71.2) | 417 (79.1) |
| 6 months | 317 (61.3) | 333 (63.2) |
| 9 months | 260 (50.3) | 269 (51.0) |
| 12 months | 220 (42.6) | 228 (43.3) |
| 15 months | 169 (32.7) | 186 (35.3) |
| 18 months | 139 (26.9) | 146 (27.7) |

The target dose (i.e., hydralazine hydrochloride and isosorbide dinitrate was 6 tablets per day; 2 tablets t.i.d; 120 mg isosorbide dinitrate and 225 mg hydralazine hydrochloride per day) was achieved at least once in 473 (89.8%) of placebo-treated patients, but in only 352 (68.1%) of the hydralazine hydrochloride and isosorbide dinitrate-treated patients. The hydralazine hydrochloride and isosorbide dinitrate-treated patients were less likely to be titrated to target doses due to the greater frequency of adverse events in this group relative to placebo. The mean number of tablets prescribed per day was consistently less in the hydralazine hydrochloride and isosorbide dinitrate-treated patients than in placebo-treated patients over the course of the trial, Table 5. For example, at 6 months, on average patients in the hydralazine hydrochloride and isosorbide dinitrate group were prescribed 29.3 mg t.i.d. of isosorbide dinitrate and 56.3 mg t.i.d. of hydralazine hydrochloride whereas patients in the placebo group were prescribed 34 mg t.i.d. of isosorbide dinitrate (placebo equivalent) and 63.8 mg t.i.d. of hydralazine hydrochloride (placebo equivalent).

TABLE 5
Mean Number of Study Drug Tablets Prescribed Per Day at Various Times

Mean (SD) # of Tablets Prescribed Per Day

| | Hydralazine | |
|---|---|---|
| | hydrochloride and | |
| | isosorbide dinitrate | Placebo |
| Time on Study | (N = 517) | (N = 527) |
| 3 months | 4.4 (2.1) | 5.0 (1.9) |
| | (n = 368) | (n = 417) |
| 6 months | 4.5 (2.0) | 5.1 (1.8) |
| | (n = 317) | (n = 333) |
| 9 months | 4.8 (1.9) | 5.2 (1.7) |
| | (n = 260) | (n = 269) |
| 12 months | 4.8 (1.9) | 5.3 (1.6) |
| | (n = 220) | (n = 228) |
| 15 months | 4.9 (1.7) | 5.3 (1.7) |
| | (n = 169) | (n = 186) |

During the course of the study, 78 (14.8%) of placebo patients and 65 (12.6%) of the hydralazine hydrochloride and isosorbide dinitrate patients received open-label treatment with long-acting nitrates, and 15 (2.8%) of placebo patients and 14 (2.7%) of the hydralazine hydrochloride and isosorbide dinitrate patients received open-label hydralazine.

### Primary Efficacy Analysis

By intention-to-treat, patients in the hydralazine hydrochloride and isosorbide dinitrate group had a significantly better clinical composite score during the course of the trial than patients in the placebo group (−0.16 vs −0.47, p=0.016 by 2-sample t-test, Table 6).

TABLE 6
Primary Efficacy Endpoint

| | Hydralazine hydrochloride and | | |
|---|---|---|---|
| Composite | isosorbide dinitrate | Placebo | |
| score | (N = 518) | (N = 532) | p-value |
| Mean (SD) | −0.16 (1.93) | −0.47 (2.04) | 0.016 |

The composite score, which is the primary endpoint, shows a statistically significant benefit for treatment when compared to control, based on the data available; p=0.016. Table 7 summarizes the results for the components score for the primary end points.

TABLE 7

| | | | |
|---|---|---|---|
| | | Hydralazine | |
| | | hydrochloride and | |
| | | Isosorbide Dinitrate | Placebo |
| | | (N = 518) | (N = 532) |
| Component | Score | n (%) | n (%) |

Death

| | | | |
|---|---|---|---|
| Yes | −3 | 32 (6.2) | 54 (10.2) |
| No | 0 | 486 (93.8) | 478 (89.8) |
| Missing | −3 | 0 (0.0) | 0 (0.0) |

First hospitalization for heart failure

| | | | |
|---|---|---|---|
| Yes | −1 | 85 (16.4) | 130 (24.4) |
| No | 0 | 420 (81.1) | 391 (73.5) |
| Missing | −1 | 13 (2.5) | 11 (2.1) |

Change in quality of life score at 6 months (or earlier) relative to baseline

| | | | |
|---|---|---|---|
| Improvement ≧10 units | 2 | 180 (38.1) | 166 (33.4) |
| Improvement ≧5 and <10 | 1 | 49 (10.4) | 56 (11.3) |
| units | | | |
| Change <5 units | 0 | 117 (22.6) | 126 (23.7) |
| Worsening ≧5 and <10 units | −1 | 46 (8.9) | 32 (6.4) |
| Worsening ≧10 units | −2 | 80 (16.9) | 117 (23.5) |
| Missing | −2 | 46 (8.9) | 35 (6.6) |

Contributing to the treatment difference on the composite score was the finding that the hydralazine hydrochloride and isosorbide dinitrate-treated group had fewer deaths (32 vs 54 for the placebo group), fewer patients with a first hospitalization for heart failure (85 vs 130), more patients with marked (≧10 unit) improvement in quality of life (180 vs 166) and fewer patients with marked (≧10 unit) worsening in quality of life (80 vs 117).

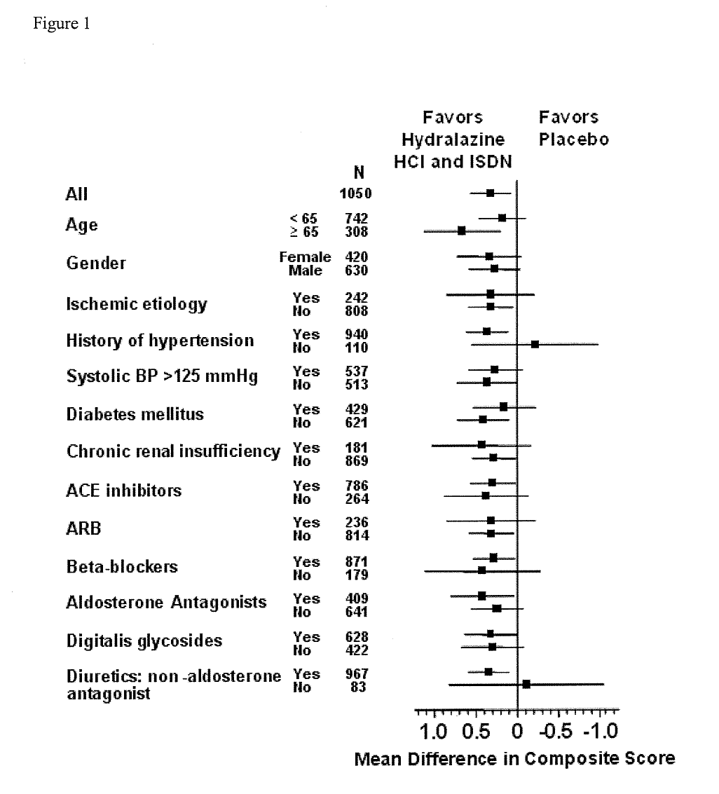

The treatment difference on the clinical composite score was seen consistently across nearly all of the subgroups examined (FIG. 1). The subgroups in which the treatment estimate did not favor hydralazine hydrochloride and isosorbide dinitrate were generally those with the fewest patients. FIG. 1 summarizes the effect of hydralazine hydrochloride and isosorbide dinitrate on composite score in subgroups (Mean±95% CI)

### Secondary Endpoints

### Mortality

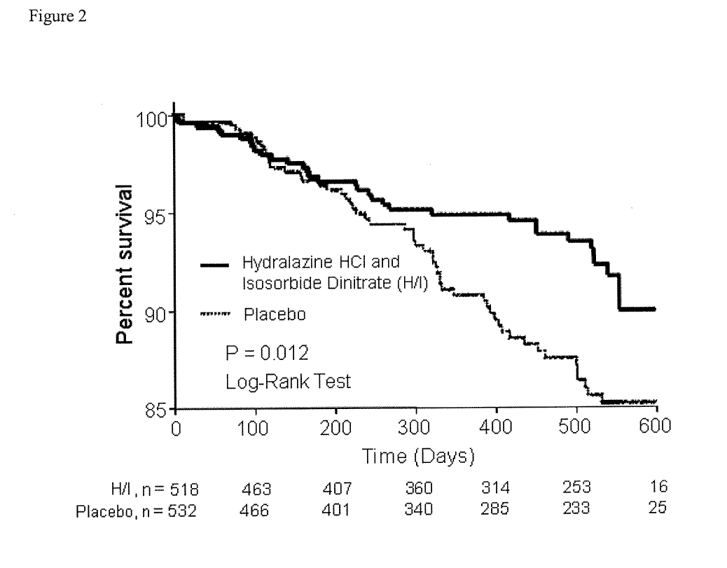

By intention to treat, 54 patients (10.2%) in the placebo group, but only 32 patients (6.2%) of the hydralazine hydrochloride isosorbide dinitrate group died during the study. This difference reflected a 43% reduction in relative risk (p=0.012; Table 8 and FIG. 2).

TABLE 8
Effect of Hydrazine and Isosorbide Dinitrate on All-Cause Mortality

| | Hydralazine | | | |
|---|---|---|---|---|
| | Hydrochloride and | | | |
| | Isosorbide Dinitrate | Placebo | Hazard ratio | Log-rank |
| n (%) | (n = 518) | (n = 532) | (95% CI) | p-value |
| All-cause | 32 (6.2%) | 54 (10.2%) | 0.57 | 0.012 |
| mortality | | | (0.37, 0.89) | |

The reduction in the overall risk of death seen in hydralazine hydrochloride and isosorbide dinitrate-treated patients was related to a reduction in heart failure deaths (i.e., sudden cardiac deaths and pump failure deaths). Other modes of death were distributed similarly across the two treatment groups (Table 9).

TABLE 9
Mode of Death

| | Hydralazine and | |
|---|---|---|
| | Isosorbide Dinitrate | Placebo |
| Category of Death (n %) | (N = 518) | (N = 532) |
| Total number of deaths | 32 (6.2) | 54 (10.2) |
| Heart failure deaths | 21 (4.1) | 42 (7.9) |
| Sudden cardiac death | 17 (3.3) | 24 (4.5) |
| Pump failure death | 4 (0.8) | 16 (3.0) |
| Death due to myocardial infarction | 0 (0.0) | 2 (0.4) |
| Non-heart failure cardiovascular death | 5 (1.0) | 3 (0.6) |
| Death due to cerebrovascular accident | 4 (0.8) | 3 (0.6) |
| Death due to other vascular event | 1 (0.2) | 0 (0.0) |
| Non-cardiovascular death | 6 (1.2) | 9 (1.7) |

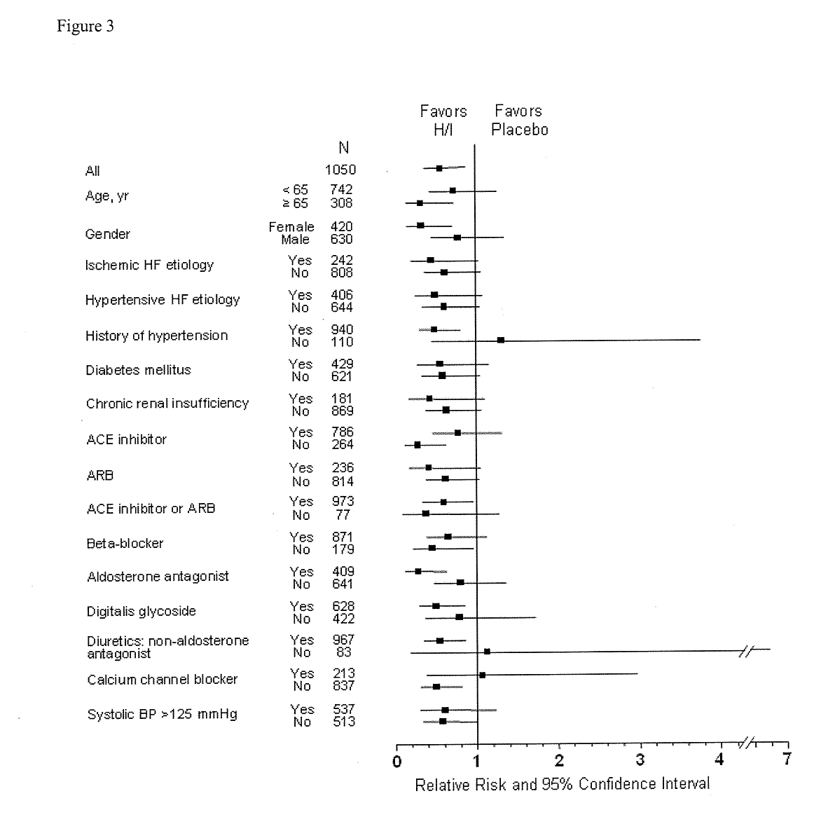

A reduction in the risk of death was seen consistently across nearly all of the subgroups examined (FIG. 3). As in the case of the primary endpoint, the subgroups in which the treatment estimate did not favor hydralazine hydrochloride and isosorbide dinitrate were generally those with the fewest patients (representing 20% or less of the patients).

### Hospitalization for Heart Failure

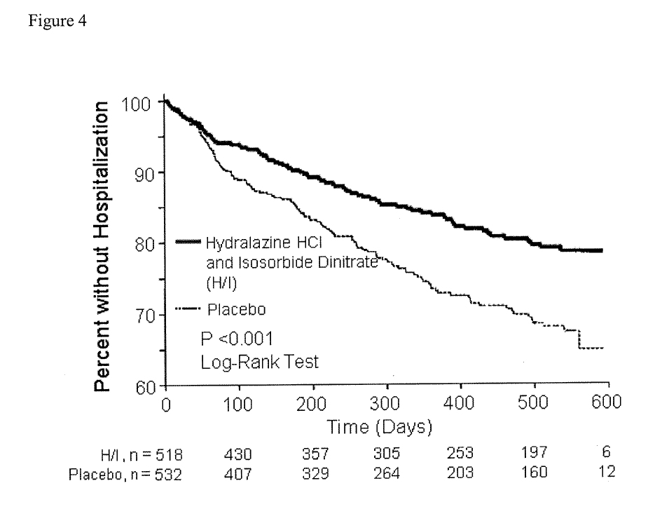

By intention to treat, 130 patients (24.4%) in the placebo group, but only 85 patients (16.4%) of the hydralazine hydrochloride and isosorbide dinitrate group were hospitalized at least once for worsening heart failure during the study. This difference reflected a 39% reduction in relative risk (p<0.001; Table 10 and FIG. 4).

TABLE 10
Effect of Hydralazine Hydrochloride and Isosorbide Dinitrate
on Risk of Hospitalization for Heart Failure

| | Hydralazine | | | |
|---|---|---|---|---|
| | hydrochloride | | | Log- |
| | and isosorbide | | Hazard | rank |
| | dinitrate | Placebo | ratio | p- |
| | (n = 518) | (n = 532) | (95% CI) | value |
| Hospitalization | 85 (16.4%) | 130 (24.4%) | 0.61 | <0.001 |
| for heart failure | | | (0.46, 0.80) | |

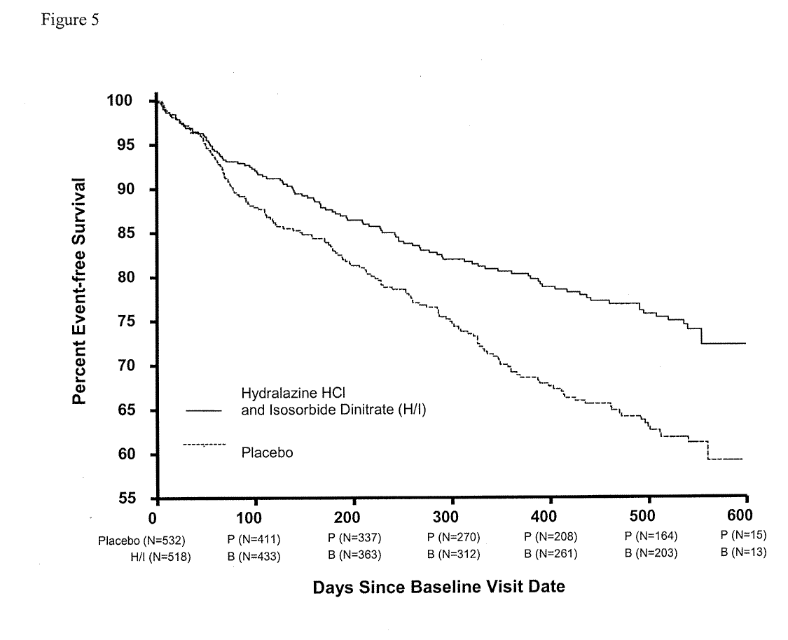

Because death and hospitalization represent competing risks, the effect of hydralazine hydrochloride and isosorbide dinitrate on the combined risk of all-cause mortality or hospitalization for heart failure was assessed even though this was not a prespecified analysis. By intention to treat, 158 patients (29.7%) in the placebo group, but only 108 patients (20.8%) in the hydralazine hydrochloride and isosorbide dinitrate group died or were hospitalized for worsening heart failure during the study. This difference reflected a 37% reduction in risk (p<0.001; Table 11 and FIG. 5).

TABLE 11
All-Cause Mortality or Hospitalization for Heart Failure

| | Hydralazine | | | |
|---|---|---|---|---|
| | hydrochloride | | | |
| | and isosorbide | | | |
| | dinitrate | Placebo | Hazard ratio | Log-rank |
| | (n = 518) | (n = 532) | (95% CI) | p-value |
| All-cause | 108 (20.8%) | 158 (29.7%) | 0.63 | <0.001 |
| mortality or | | | (0.49, 0.81) | |
| hospitalization | | | | |
| for heart | | | | |
| failure | | | | |

### Quality of Life

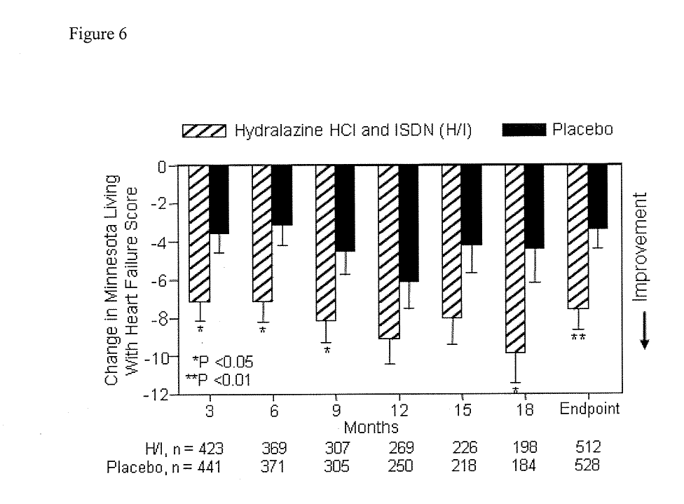

When compared with placebo, the hydralazine hydrochloride and isosorbide dinitrate-treated patients experienced greater improvements in quality of life, as assessed by the Minnesota Living with Heart Failure questionnaire, at most visits during the course of the study relative to baseline (FIG. 6, Tables 12, 13). [A decrease in score denotes improvement in quality of life; endpoint refers to last available measurement.] The improvement was seen primarily in the physical domain of the questionnaire.

TABLE 12
Change in Overall, Emotional, and Physical Scores in Minnesota
Living with Heart Failure Questionnaire at Six Months

| | Hydralazine | | |
|---|---|---|---|
| | hydrochloride | | |
| | and isosorbide | | |
| | dinitrate | Placebo | |
| | (N = 518) | (N = 532) | p-value |
| Overall score | | | |
| n | 512 | 528 | |
| Baseline Mean (SD) | 50.9 (24.9) | 50.8 (25.5) | |
| Difference Mean (SD) | −7.6 (22.6) | −3.4 (22.7) | 0.003 |
| Physical score | | | |
| n | 512 | 528 | |
| Baseline Mean (SD) | 22.1 (11.0) | 22.0 (11.2) | |
| Difference Mean (SD) | −3.5 (10.5) | −1.4 (10.6) | 0.002 |
| Emotional score | | | |
| n | 512 | 528 | |
| Baseline Mean (SD) | 10.4 (7.8) | 10.4 (7.8) | |
| Difference Mean (SD) | −1.3 (6.8) | −0.7 (6.5) | 0.129 |

TABLE 13
Change in Overall, Emotional and Physical Scores in Minnesota
Living with Heart Failure Questionnaire at Endpoint*

| | Hydralazine | | |
|---|---|---|---|
| | hydrochloride | | |
| | and isosorbide | | |
| | dinitrate | Placebo | |
| | (N = 518) | (N = 532) | p-value |
| Overall score | | | |
| n | 369 | 371 | |
| Baseline Mean (SD) | 52.5 (24.5) | 51.1 (26.0) | |
| Difference Mean (SD) | −7.1 (20.6) | −3.1 (21.3) | 0.011 |
| Physical score | | | |
| n | 369 | 371 | |
| Baseline Mean (SD) | 22.7 (10.9) | 21.9 (11.3) | |
| Difference Mean (SD) | −3.0 (9.7) | −1.3 (9.7) | 0.017 |
| Emotional score | | | |
| n | 369 | 370 | |
| Baseline Mean (SD) | 10.8 (7.7) | 10.5 (7.9) | |
| Difference Mean (SD) | −1.5 (6.2) | −0.5 (6.4) | 0.036 |

*Endpoint defined as last measurement on study.

### Other Secondary Endpoints

### Total Number of Hospitalizations and Hospital Days

When compared with placebo, patients in the hydralazine hydrochloride and isosorbide dinitrate group had fewer hospitalizations for heart failure and spent fewer days in the hospital for heart failure, p<0.01 (Tables 14, 15). Compared to placebo, patients in the hydralazine hydrochloride and isosorbide dinitrate group also had fewer hospitalizations and spent fewer days in the hospital for any reason. Hospitalizations in the hydralazine hydrochloride and isosorbide dinitrate group were shorter than in the placebo group, whether they were for heart failure or for any reason.

TABLE 14
Hospitalizations for Heart Failure

| | Hydralazine | | |
|---|---|---|---|
| | hydrochloride | | |
| | and isosorbide | | |
| | dinitrate | Placebo | |
| | N = 518 | N = 532 | p-value |
| Total number of hospitalizations | 173 | 251 | |
| for heart failure | | | |
| Mean number of hospitalizations | 0.3 | 0.5 | 0.002 |
| for heart failure per patient | | | |
| Hospitalizations by frequency | | | 0.008 |
| 0 | 433 | 402 | |
| 1 | 44 | 69 | |
| 2 | 20 | 38 | |
| 3 | 10 | 7 | |
| ≧4 | 11 | 16 | |
| Total number of hospital | 1167 | 1995 | |
| days for heart failure | | | |
| Mean number of days in the | 2.3 | 3.8 | 0.001 |
| hospital for heart failure per | | | |
| patient | | | |
| Mean number of days per | 6.7 | 7.9 | |
| hospitalization for heart failure | | | |

TABLE 15
Hospitalizations for Any Reason

| | Hydralazine | | |
|---|---|---|---|
| | hydrochloride | | |
| | and isosorbide | | |
| | dinitrate | Placebo | |
| | N = 518 | N = 532 | p-value |
| Total number of hospitalizations | 435 | 559 | |
| for any reason | | | |
| Mean number of hospitalizations | 0.8 | 1.1 | 0.14 |
| for any reason per patient | | | |
| Hospitalizations by frequency | | | 0.17 |
| 0 | 316 | 311 | |
| 1 | 99 | 85 | |
| 2 | 50 | 59 | |
| 3 | 24 | 30 | |
| ≧4 | 29 | 47 | |
| Total number of hospital | 2626 | 3902 | |
| days for any reason | | | |
| Mean number of days in the | 5.1 | 7.3 | 0.11 |
| hospital for any reason per patient | | | |
| Mean number of days per | 6.0 | 7.0 | |
| hospitalization for any reason | | | |

The number of patients with an adjudicated need for heart transplantation was similar in the two treatment groups (3 in the hydralazine hydrochloride and isosorbide dinitrate group and 5 in the placebo group), p=0.726.

There was no difference between placebo group and the hydralazine hydrochloride and isosorbide dinitrate group in the number of emergency room visits or unscheduled office/clinic visits for heart failure.

### Safety Results

Table 16 displays the proportion of patients with at least one adverse event, the number with at least one serious adverse event (other than an endpoint event) and the number who permanently discontinued treatment with the study drug due to an adverse event.

TABLE 16
Overview of Patients with Adverse Events

| | Hydralazine | |
|---|---|---|
| | hydrochloride and | |
| | isosorbide dinitrate | Placebo |
| Adverse Event Category (#, %) | n = 517 | n = 527 |
| Patients with at least one adverse | 475 (91.9%) | 432 (82.0%) |
| event | | |
| Patients with at least one serious | 181 (35.0%) | 183 (34.7%) |
| adverse event (excluding endpoint | | |
| events) | | |
| Patients who permanently | 109 (21.1%) | 63 (12.0%) |
| discontinued study drug due to | | |
| adverse events | | |

### Adverse Events Regardless of Relationship to Study Drug

Table 17 lists the number of patients with an adverse event that occurred in at least 2% of patients in either treatment group, whether or not patients were taking the study medication. In general, adverse events related to systemic vasodilation (headache, dizziness, hypotension, tachycardia and sinusitis [sinus congestion]), or reflecting gastrointestinal distress (nausea and vomiting) were more frequent in the hydralazine hydrochloride and isosorbide dinitrate-treated than placebo-treated patients. In contrast, adverse events related to worsening heart failure (heart failure, dyspnea, increased cough and peripheral edema) were more common in placebo-treated patients than in the hydralazine hydrochloride and isosorbide dinitrate-treated patients.

Four events (nausea, heart failure, hypotension and sinusitis) were significant at the 0.05 level; headache and dizziness were significant at the 0.0001 level.

TABLE 17
Adverse Events Occurring in ≧2% of Patients in Either Group

| | Hydralazine hydrochloride | |
|---|---|---|
| | and isosorbide | Placebo |
| | dinitrate (n = 517) | (n = 527) |
| Adverse Event* | n (%) | n (%) |
| Headache | 256 (49.5) | 111 (21.1) |
| Dizziness | 165 (31.9) | 72 (13.7) |
| Pain | 84 (16.2) | 85 (16.1) |
| Chest pain | 81 (15.7) | 80 (15.2) |
| Infection | 70 (13.5) | 67 (12.7) |
| Asthenia | 70 (13.5) | 59 (11.2) |
| Dyspnea | 65 (12.6) | 92 (17.5) |
| Nausea | 50 (9.7) | 32 (6.1) |
| Heart failure | 49 (9.5) | 80 (15.2) |
| Bronchitis | 43 (8.3) | 34 (6.5) |
| Hypotension | 41 (7.9) | 23 (4.4) |
| Hypertension | 33 (6.4) | 33 (6.3) |
| Accidental injury | 29 (5.6) | 36 (6.8) |
| Increased cough | 27 (5.2) | 41 (7.8) |
| Gout | 27 (5.2) | 32 (6.1) |
| Diarrhea | 27 (5.2) | 30 (5.7) |
| Peripheral edema | 25 (4.8) | 37 (7.0) |
| Abdominal pain | 25 (4.8) | 35 (6.6) |
| Back pain | 24 (4.6) | 28 (5.3) |
| Insomnia | 23 (4.4) | 24 (4.6) |
| Syncope | 23 (4.4) | 20 (3.8) |
| Sinusitis | 22 (4.3) | 9 (1.7) |
| Anemia | 21 (4.1) | 26 (4.9) |
| Ventricular tachycardia | 21 (4.1) | 14 (2.7) |
| Hyperglycemia | 20 (3.9) | 18 (3.4) |
| Palpitations | 20 (3.9) | 14 (2.7) |
| GI disorder | 20 (3.9) | 14 (2.7) |
| Urinary tract infection | 19 (3.7) | 26 (4.9) |
| Pneumonia | 19 (3.7) | 21 (4.0) |
| Rhinitis | 19 (3.7) | 14 (2.7) |
| Constipation | 18 (3.5) | 28 (5.3) |
| Depression | 18 (3.5) | 25 (4.7) |
| Paresthesia | 18 (3.5) | 12 (2.3) |
| Vomiting | 18 (3.5) | 10 (1.9) |
| Pharyngitis | 17 (3.3) | 24 (4.6) |
| Dyspepsia | 16 (3.1) | 24 (4.6) |
| Blurred vision | 16 (3.1) | 7 (1.3) |
| Hypokalemia | 15 (2.9) | 18 (3.4) |
| Hyperlipemia | 15 (2.9) | 10 (1.9) |
| Arrhythmia | 14 (2.7) | 20 (3.8) |
| Abnormal kidney function | 14 (2.7) | 7 (1.3) |
| Pruritus | 13 (2.5) | 13 (2.5) |
| Hyperkalemia | 12 (2.3) | 20 (3.8) |
| Flu syndrome | 12 (2.3) | 18 (3.4) |
| Asthma | 12 (2.3) | 15 (2.8) |
| Edema | 12 (2.3) | 14 (2.7) |
| Rash | 12 (2.3) | 14 (2.7) |
| Nausea vomiting | 11 (2.1) | 11 (2.1) |
| Dehydration | 11 (2.1) | 11 (2.1) |
| Cellulitis | 11 (2.1) | 9 (1.7) |
| Tachycardia | 11 (2.1) | 6 (1.1) |
| Diabetes mellitus | 10 (1.9) | 15 (2.8) |
| Lung disorder | 10 (1.9) | 15 (2.8) |
| Cramps leg | 10 (1.9) | 12 (2.3) |
| Hypoglycemia | 10 (1.9) | 11 (2.1) |
| Acute kidney failure | 8 (1.5) | 15 (2.8) |
| Increased weight | 8 (1.5) | 13 (2.5) |
| Cerebrovascular accident | 7 (1.4) | 13 (2.5) |
| Increased sputum | 6 (1.2) | 11 (2.1) |

*A patient can have more than one event or type of event; each patient is counted once in each category.

### Serious Adverse Events Regardless of Relationship to Study Drug

Table 18 lists the numbers of patients with a serious adverse event that occurred in at least 1% of the patients in either treatment group, whether or not patients were taking the study medication. In general, adverse events related to systemic vasodilation or tachycardia (chest pain, ventricular tachycardia, syncope, arrhythmia, hypotension and dizziness) were somewhat more common in the hydralazine hydrochloride and isosorbide dinitrate-treated patients, whereas adverse events related to worsening heart failure or other major clinical events (heart failure, dyspnea, cerebrovascular accident and myocardial infarction) were more common in placebo-treated patients. Only the incidence of reports of heart failure was significant (p<0.001).

TABLE 18
Serious Adverse Events Occurring in ≧1% of Patients in Either Group

| | Hydralazine hydrochloride | |
|---|---|---|
| | and isosorbide dinitrate | Placebo |
| | N = 517 | N = 527 |
| Serious Adverse Event* | n (%) | n (%) |
| Chest pain | 33 (6.4) | 29 (5.5) |
| Heart failure | 16 (3.1) | 41 (7.8) |
| Ventricular tachycardia | 14 (2.7) | 8 (1.5) |
| Pneumonia | 12 (2.3) | 8 (1.5) |
| Syncope | 11 (2.1) | 8 (1.5) |
| Dyspnea | 10 (1.9) | 12 (2.3) |
| Arrhythmia | 9 (1.7) | 7 (1.3) |
| Hypotension | 8 (1.5) | 3 (0.6) |
| Cerebrovascular accident | 7 (1.4) | 13 (2.5) |
| Heart arrest | 7 (1.4) | 9 (1.7) |
| Dizziness | 7 (1.4) | 0 (0.0) |
| Diabetes mellitus | 6 (1.2) | 5 (0.9) |
| Cellulitis | 6 (1.2) | 2 (0.4) |
| Acute kidney failure | 5 (1.0) | 8 (1.5) |
| Lung disorder | 5 (1.0) | 6 (1.1) |
| Infection | 5 (1.0) | 5 (0.9) |
| Angina pectoris | 5 (1.0) | 5 (0.9) |
| Hyperglycemia | 5 (1.0) | 5 (0.9) |
| Hypoglycemia | 5 (1.0) | 5 (0.9) |
| Dehydration | 5 (1.0) | 4 (0.8) |
| Anemia | 5 (1.0) | 3 (0.6) |
| Bronchitis | 5 (1.0) | 3 (0.6) |
| Coronary artery disease | 5 (1.0) | 2 (0.4) |
| Cerebral ischemia | 5 (1.0) | 1 (0.2) |
| Myocardial infarction | 4 (0.8) | 9 (1.7) |
| Abdominal pain | 4 (0.8) | 8 (1.5) |
| Hypertension | 4 (0.8) | 7 (1.3) |
| Accidental injury | 3 (0.6) | 8 (1.5) |

*Excludes endpoint events such as death or hospitalization for heart failure. A patient can have more than one event or type of event; each patient is counted only once in each category.

### Adverse Events Leading to Permanent Withdrawal of Study Drug

Table 19 lists the number of patients with an adverse event that led to the permanent withdrawal of the study drug. The adverse events that were seen most frequently in the Hydralazine hydrochloride and isosorbide dinitrate-treated group were also the most common cause of withdrawal of the study drug, e.g., headache, dizziness, asthenia, chest pain, nausea and hypotension.

TABLE 19
Adverse Events Occurring in ≧0.4% of Patients in Either Group and
Leading to Permanent Discontinuation of Study Drug

| | Hydralazine hydrochloride | |
|---|---|---|
| | and isosorbide dinitrate | Placebo |
| | N = 517 | N = 527 |
| Adverse Event* | n (%) | n (%) |
| Headache | 38 (7.4) | 4 (0.8) |
| Dizziness | 19 (3.7) | 4 (0.8) |
| Asthenia | 12 (2.3) | 1 (0.2) |
| Chest pain | 8 (1.5) | 2 (0.4) |
| Nausea | 8 (1.5) | 2 (0.4) |
| Hypotension | 7 (1.4) | 3 (0.6) |
| Pain | 4 (0.8) | 1 (0.2) |
| Heart failure | 3 (0.6) | 4 (0.8) |
| Heart arrest | 3 (0.6) | 3 (0.6) |
| Paresthesia | 3 (0.6) | 0 (0.0) |
| Diarrhea | 2 (0.4) | 2 (0.4) |
| Confusion | 2 (0.4) | 2 (0.4) |
| Chills | 2 (0.4) | 1 (0.2) |
| Malaise | 2 (0.4) | 1 (0.2) |
| Abdominal pain | 2 (0.4) | 1 (0.2) |
| Kidney failure | 2 (0.4) | 1 (0.2) |
| Ventricular fibrillation | 2 (0.4) | 0 (0.0) |
| Palpitations | 2 (0.4) | 0 (0.0) |
| Syncope | 2 (0.4) | 0 (0.0) |
| Nausea vomiting | 2 (0.4) | 0 (0.0) |
| Abnormal kidney function | 2 (0.4) | 0 (0.0) |
| Dyspnea | 1 (0.2) | 4 (0.8) |
| Cerebrovascular accident | 1 (0.2) | 3 (0.6) |
| Constipation | 1 (0.2) | 3 (0.6) |
| Dyspepsia | 1 (0.2) | 2 (0.4) |
| Myocardial infarction | 0 (0.0) | 4 (0.8) |
| Rash | 0 (0.0) | 3 (0.6) |
| Rectal hemorrhage | 0 (0.0) | 2 (0.4) |
| Hypoglycemia | 0 (0.0) | 2 (0.4) |

*Excludes endpoint events such as death or hospitalization for heart failure. A patient can have more than one event or type of event; each patient is counted only once in each category.

### Other Safety Topics

There was little change in heart rate during the trial, and heart rate responses did not differ between the two treatment groups. In contrast, both systolic and diastolic blood pressure in the hydralazine hydrochloride and isosorbide dinitrate-treated patients were significantly lower than in placebo-treated patients (Table 20).

TABLE 20
Mean Change in Heart Rate, Systolic Blood Pressure
and Diastolic Blood Pressure (BP)

| | Change in | | Change in | | Change in | |
|---|---|---|---|---|---|---|
| | Heart Rate | | Systolic BP | | Diastolic BP | |
| | (bpm) | | (mmHg) | | (mmHg) | |
| | Hydralazine | | Hydralazine | | Hydralazine | |
| | hydrochloride | | hydrochloride | | hydrochloride | |
| Time on | and isosorbide | | and isosorbide | | and isosorbide | |
| Study | dinitrate | Placebo | dinitrate | Placebo | dinitrate | Placebo |
| 3 Months | 1.3 | 1.3 | −3.2* | 1.1 | −3.4* | 0.3 |
| | n = 434 | n = 468 | n = 436 | n = 469 | n = 436 | n = 467 |
| 6 Months | 1.3 | 0.0 | −1.9* | 1.2 | −2.4* | 0.8 |
| | n = 387 | n = 375 | n = 389 | n = 375 | n = 389 | n = 375 |
| 9 Months | 2.3 | 1.4 | −4.7* | 0.4 | −3.3* | 0.2 |
| | n = 312 | n = 305 | n = 313 | n = 304 | n = 313 | n = 304 |
| 12 Months | 1.5 | 0.7 | −3.1* | 2.0 | −2.8* | 0.9 |
| | n = 271 | n = 257 | n = 276 | n = 258 | n = 276 | n = 258 |
| 15 Months | 1.6 | 1.7 | −3.1* | 0.9 | −2.9* | 0.7 |
| | n = 221 | n = 217 | n = 225 | n = 217 | n = 225 | n = 217 |
| 18 Months | 3.0 | 0.4 | −3.4* | 1.2 | −3.0* | 0.3 |
| | n = 196 | n = 175 | n = 197 | n = 175 | n = 197 | n = 175 |

*p < 0.05 comparison of Hydralazine hydrochloride and isosorbide dinitrate to placebo, two-sample t-test

Six hydralazine hydrochloride and isosorbide dinitrate-treated patients and one placebo-treated patient experienced an adverse event classified as angioedema. The events were identified as serious in two hydralazine hydrochloride and isosorbide dinitrate-treated patients and no placebo-treated patients; these two serious events are described below.

The first patient experienced facial and lip swelling five days after the initiation of hydralazine hydrochloride and isosorbide dinitrate He was treated in an emergency room with diphenhydramine, dexamethasone, and methylprednisolone and discharged after improvement was noted. Study drug was discontinued.

The second patient was randomized to hydralazine hydrochloride and isosorbide dinitrate and approximately seven months later experienced shortness of breath and swelling of the lips and tongue following ingestion of his morning medications; he then became unresponsive. Emergency medical services administered fluids and diphenhydramine, resulting in return of his mental status. In the Emergency Room he was treated with diphenhydramine and methylprednisolone; the lip and tongue swelling improved, and he was discharged and advised to discontinue his angiotensin-converting enzyme inhibitor and refrain from alcohol. No action was taken with respect to study drug administration.

Patients in the treatment group had a slight but significant blood pressure lowering affect at 6 months. Systolic blood pressure was reduced by 1.9 mm Hg as compared with an increase of 1.2 mm Hg in the placebo group (p=0.02). The diastolic blood pressure was reduced by 2.4 mm Hg, as compared to an increase of 0.8 mm Hg in the placebo group (p=0.001). Heart rate was unchanged.

Heart failure exacerbations, evaluated as either serious adverse events (SAEs) or adverse events (AEs) showed a statistically significant benefit for treatment when compared to control. 12.8% of the patients in the control group had an SAE associated with exacerbation of heart failure; in contrast, 8.7% of the patients in the treatment arm had such an SAE. (p=0.04). 7.0% of the patients in the control group had an AE associated with exacerbation of heart failure, whereas 3/1% of the patients in the treatment group had such an AE, p=0.005. Overall SAEs are favorable for treatment relative to control.

### Left Ventricular Ejection Fraction and BNP

In the trial, baseline and 6-month echocardiograms were performed in 823 patients. Echocardiograms were digitized and analyzed blindly in an independent core laboratory (Bioimaging). B-type natriuretic peptide (BNP) was also measured at baseline and at 6 months.

Left ventricular ejection fraction increased by 2.14% units in the patients administered the combination of isosorbide dinitrate and hydralazine vs. 0.77% units in the patients administered placebo (p=0.005). Left ventricular internal diastolic dimension decreased by 0.22 cm in the patients administered the combination of isosorbide dinitrate and hydralazine and by 0.01 cm in the patients administered placebo (p=0.01). BNP at baseline (145 pg/ml isosorbide dinitrate and hydralazine group, 167 pg/ml in the placebo group) was reduced at 6 months by 21 pg/ml in isosorbide dinitrate and hydralazine group and 5 pg/ml in the placebo group (p=0.05).

### SUMMARY AND CONCLUSIONS

The administration of a combination of hydralazine hydrochloride and isosorbide dinitrate for the treatment of heart failure in a patient in need thereof results in the follow:

The long-term administration of a combination of hydralazine hydrochloride and isosorbide dinitrate to patients with moderate-to-severe heart failure generally treated with angiotensin converting enzyme inhibitors, β-adrenergic antagonists, angiotensin II antagonists, aldosterone antagonists, cardiac glucosides (digitalis), and diuretic compounds was associated with a 43% reduction in the relative risk of death (p=0.012).

The survival benefit of patients administered a combination of hydralazine hydrochloride and isosorbide dinitrate was accompanied by a significant improvement in the primary endpoint of the trial (p=0.016), which combined information about the occurrence of death, first hospitalization for heart failure and change in quality of life into a single variable.

The long-term administration of hydralazine hydrochloride and isosorbide dinitrate to patients reduced the relative risk of hospitalization for heart failure by 39% (p<0.001). A combination of hydralazine hydrochloride and isosorbide dinitrate also reduced the combined relative risk of death or hospitalization for heart failure by 37% (p<0.001).

When compared with placebo, patients in the hydralazine hydrochloride and isosorbide dinitrate group had fewer hospitalizations for heart failure and spent fewer days in the hospital for heart failure, (both p<0.01). Patients in the hydralazine hydrochloride and isosorbide dinitrate group also had fewer hospitalizations and spent fewer days in the hospital for any reason, but the differences were not significant. Hospitalizations in the hydralazine hydrochloride and isosorbide dinitrate group were shorter than in the placebo group, whether they were for heart failure or for any reason.

Hydralazine hydrochloride and isosorbide dinitrate-treated patients experienced greater improvements in quality of life, as assessed by the Minnesota Living with Heart Failure questionnaire, at most visits during the course of the study.

Worsening heart failure as an adverse event was reported less frequently in patients in the hydralazine hydrochloride and isosorbide dinitrate group than those in the placebo group (9.5% vs 15.2%). Worsening heart failure as a serious adverse event was reported less frequently in patients in the hydralazine hydrochloride and isosorbide dinitrate group than those in the placebo group (3.1% vs 7.8%).

The clinical benefits of the combination of hydralazine hydrochloride and isosorbide dinitrate were associated with a persistent decrease in systolic and diastolic blood pressure, which did not become attenuated over time.

The disclosure of each patent, patent application and publication cited or described in the present specification is hereby incorporated by reference herein in its entirety.

Although the invention has been set forth in detail, one skilled in the art will appreciate that numerous changes and modifications can be made to the invention without departing from the spirit and scope thereof.

What is claimed is:

1. A method to prolong time to hospitalization for heart failure in a patient in need thereof comprising administering to the patient hydralazine hydrochloride in an amount of 30 milligrams to 400 milligrams and isosorbide dinitrate in an amount of 10 milligrams to 200 milligrams.

2. The method of claim 1, comprising administering (i) 37.5 mg hydralazine hydrochloride and 20 milligrams isosorbide dinitrate or (ii) 75 mg hydralazine hydrochloride and 40 milligrams isosorbide dinitrate; wherein the hydralazine hydrochloride and the isosorbide dinitrate are administered separately or as components of the same composition.

3. The method of claim 1, further comprising administering at least one compound selected from the group consisting of an angiotensin converting enzyme inhibitor, a β-adrenergic antagonist, an angiotensin II antagonist, an aldosterone antagonist, a cardiac glucoside and a diuretic compound.

4. The method of claim 1, wherein the patient is categorized as New York Heart Association heart failure functional classification I, II, III or IV.

5. A method to reduce the number of days a patient spends in the hospital for heart failure or to reduce the number of hospital admissions for heart failure in a patient in need thereof comprising administering to the patient hydralazine hydrochloride in an amount of 30 milligrams to 400 milligrams per day and isosorbide dinitrate in an amount of 10 milligrams to 200 milligrams per day.

6. The method of claim 5, comprising administering (i) 37.5 mg hydralazine hydrochloride and 20 milligrams isosorbide dinitrate or (ii) 75 mg hydralazine hydrochloride and 40 milligrams isosorbide dinitrate; wherein the hydralazine hydrochloride and the isosorbide dinitrate are administered separately or as components of the same composition.

7. The method of claim 5, further comprising administering at least one compound selected from the group consisting of an angiotensin converting enzyme inhibitor, a β-adrenergic antagonist, an angiotensin II antagonist, an aldosterone antagonist, a cardiac glucoside and a diuretic compound.

8. The method of claim 5, wherein reducing the number of days a patient spends in the hospital for heart failure is for one or more hospital admissions.

9. The method of claim 5, wherein the patient is categorized as New York Heart Association heart failure functional classification I, II, III or IV.

10. A method for (a) prolonging time to hospitalization for heart failure; (b) prolonging time to first hospitalization for heart failure; (c) reducing the number of days a patient spends in the hospital for heart failure; (d) reducing the total number of days a patient spends in the hospital for heart failure for two or more hospital stays; (e) reducing the number of hospital admissions for heart failure; (f) reducing mortality and reduce hospitalizations for heart failure; (g) increasing left ventricular ejection fraction in a heart failure patient; (h) treating a sexual dysfunction (j) treating a headache in a heart failure patient by administering a non-steroidal antiinflammatory compound; (k) treating a heart failure patient who has a history of hypertension but who is not currently diagnosed with hypertension; (l) improving the quality of life in a heart failure patient based on the Minnesota Living with heart failure questionnaire; (m) decreasing levels of B-type natriuretic peptide; (n) treating hypertension in a heart failure patient; (o) lowering blood pressure in a heart failure patient; (p) treating labile hypertension; (q) treating idiopathic hypertension; (r) increasing patient compliance with medication dosing in a heart failure patient; (s) treating hypertension in a patient with a dilated heart; (t) treating ischemic disease and/or coronary artery disease; or (u) reducing cardiomegaly in a patient in need thereof comprising administering to the patient a therapeutically effective amount of (i) a hydralazine compound or a pharmaceutically acceptable salt thereof and (ii) isosorbide dinitrate and/or isosorbide mononitrate.

11. The method of claim 10, comprising administering a therapeutically effective amount of a hydralazine hydrochloride and isosorbide dinitrate; wherein the hydralazine hydrochloride and the isosorbide dinitrate are administered separately or as components of the same composition.

12. The method of claim 11, comprising administering hydralazine hydrochloride in an amount of about 30 milligrams to about 400 milligrams per day and isosorbide dinitrate in an amount of about 10 milligrams to about 200 milligrams per day.

13. The method of claim 11, comprising administering hydralazine hydrochloride in an amount of about 225 milligrams per day and isosorbide dinitrate in an amount of about 120 milligrams per day.

14. The method of claim 11, comprising administering hydralazine hydrochloride in an amount of about 112.5 milligrams once or twice per day and isosorbide dinitrate in an amount of about 60 milligrams once or twice per day.

15. The method of claim 11, comprising administering hydralazine hydrochloride in an amount of about 75 milligrams once, twice or three times per day and isosorbide dinitrate in an amount of about 40 milligrams once, twice or three times per day.

16. The method of claim 11, comprising administering hydralazine hydrochloride in an amount of about 37.5 milligrams once, twice or three times per day and isosorbide dinitrate in an amount of about 20 milligrams once, twice or three times per day.

17. The method of claim 10, further comprising administering at least one compound selected from the group consisting of an angiotensin converting enzyme inhibitor, a β-adrenergic antagonist, an angiotensin II antagonist, an aldosterone antagonist, a cardiac glucoside and a diuretic compound.

18. The method of claim 10, further comprising administering captopril, enalapril, lisinopril, metoprolol, or nebivolol.

19. The method of claim 10, wherein the patient is categorized as New York Heart Association heart failure functional classification I, II, III or IV.

20. The method of claim 10, wherein the patient is a black patient.
